(19) United States
(12) Patent Application Publication
(10) Pub. No.: US 2018/0087024 A1
(43) Pub. Date: Mar. 29, 2018

(54) GENETICALLY ENGINEERED C1-UTILIZING MICROORGANISMS AND PROCESSES FOR THEIR PRODUCTION AND USE

(71) Applicant: National Research Council of Canada, Ottawa (CA)

(72) Inventors: Martin G. Lamarche, Laval (CA); Jonathan Perreault, Montreal (CA); Carlos Miguez, Beaconsfield (CA); Melanie Arbour, Ste-Marthe-sur-le-Lac (CA); Young Jun Choi, Pointe-Claire (CA)

(73) Assignee: National Research Council of Canada, Ottawa, ON (CA)

(21) Appl. No.: 15/566,579

(22) PCT Filed: Apr. 15, 2016
(86) PCT No.: PCT/CA2016/050439

§ 371 Date: Oct. 13, 2017

Related U.S. Application Data
(60) Provisional application No. 62/148,350, filed on Apr. 16, 2015.

(51) Int. Cl. C12N 1/32; C07K 14/195; C12N 15/52; C12N 9/88; C12N 9/04; C12R 1/26

(52) U.S. Cl. CPC C12N 1/32; C07K 14/195; C12N 15/52; C12R 1/26; C12Y 604/01001; C12N 9/0006; C12N 9/88

(57) ABSTRACT

Described are genetically engineered C1-utilizing bacteria for the preparation of dicarboxylic acids, e.g. succinic acid. For instance, the bacteria comprise a mutation in a gene encoding a tricarboxylic acid cycle (TCA) succinate dehydrogenase (Sdh), preferably a mutation which inactivates or reduces Sdh's activity. Processes for the production of the modified bacteria as well as their use in the preparation of succinic acid on a C1-compound as carbon source are also discussed.

### SEQUENCE LISTING

The instant application contains a Sequence Listing which has been submitted in ASCII format via EFS-Web and is hereby incorporated by reference in its entirety. Said ASCII copy, created on Apr. 11, 2016, is named Sequence_Listing.txt.

### TECHNICAL FIELD

The present description relates to genetically modified C1-utilizing microorganisms like bacteria, processes for producing them and their use in the preparation of dicarboxylic acids, more particularly succinic acid. The description further relates to genetically engineered methylotroph or methanotroph bacteria, processes for their preparation and their use in the production of succinic acid.

### BACKGROUND

Succinic acid is a natural four carbon dicarboxylic acid. It can be found in all living cells: plant, animal or bacteria. Its name is derived from the latin succinum, which means amber, the historical source of succinic acid, originally known as the Spirit of amber¹. This organic acid has multiple uses in various industries: food and drink aromatization, chemical intermediary for coloring agents, perfumes, lacquer, alkyde resins and plasticizers as well as water cooling systems and even metal treatment. Succinic acid belongs to the twelve most valuable building block chemicals². This acid can replace maleic acid (or anhydric maleic) in the production of basic chemicals such as 1,4-butanediol (BDO) and plasticizers.

Until recently, synthesis of succinic acid at an industrial scale involved catalytic hydrogenation of maleic acid, derived from benzene or butane³. The cost of succinic acid produced in this way is relatively high because it is linked to the cost of the corresponding raw material: fossil fuels. In addition, this situation causes unpredictable fluctuation in the cost of the raw material, another undesirable factor for the industry. While raw material originating from agriculture presents many advantages freeing in part succinic acid production from fossil fuels, it is still controversial to some extent because it requires the use of cultivated land and resources, which could rather be used for food production.

There is thus a need for a third generation organism, which could produce succinic acid from more sustainable and/or economical raw material.

### SUMMARY

According to one aspect, a genetically engineered C1-utilizing bacterium is described, wherein the bacterium is modified to disrupt a gene encoding a tricarboxylic acid (TCA) cycle succinate dehydrogenase (Sdh) or a subunit thereof. In one embodiment, the bacterium is a serine cycle methylotroph bacterium, for example, from the genera Burkholderia, Fulvimarina, Granulibacter, Hyphomicrobium, Methylibium, Methylobacterium, Ruegeria, preferably Methylobacterium. In another embodiment, the bacterium is a serine cycle methanotroph bacterium, for example, from the genera Methanomonas, Methylocystis, Methylocapsa, Methylocella, Methylococcus and Methylosinus, preferably Methylosinus. In one embodiment, the bacterium is modified by the knock out, knockdown or deletion of an sdh gene, for example an sdhA gene.

In an embodiment, the bacterium as herein defined is further modified to inactivate or reduce the activity of a protein involved in polyhydroxyalkanoate (PHA) biosynthesis and/or polyhydroxyalkanoate granule homeostasis, for example by the knockout, knockdown or deletion of a gene encoding the protein (e.g. a phasin, a PHA synthase). In one embodiment, the polyhydroxyalkanoate is a poly-β-hydroxybutyric acid (PHB). In some embodiments, the protein involved in polyhydroxyalkanoate biosynthesis and/or polyhydroxyalkanoate granule homeostasis is a Granule-Associated Protein (GAP), a phasin, a PHB synthase, Gap11, Gap 20, PhaC, or PhaR.

In a further embodiment, the bacterium further comprises the overexpression of a TCA cycle succinyl-CoA synthetase, for example SucC and/or SucD. In one embodiment, the overexpression comprises the insertion of a P_{mxaF}sucCD DNA fragment into a chromosome.

According to a further embodiment, the bacterial strain is as defined in any of the aforementioned embodiments and further comprises one or more of the following: (a) overexpression of one or more serine-cycle enzymes through modifications of their respective genes, for instance modifications to glyA, eno and/or mdh genes, encoding respectively serine hydroxymethyltransferase, enolase and malate dehydrogenase enzymes; (b) heterologous expression of one or more genes involved in succinic acid production, e.g. pyc (encoding a pyruvate carboxylase), ppc (encoding a phosphoenol pyruvate carboxylase), and/or icl (encoding isoctirate lyase); (c) incorporation of genetic switch(es), e.g. sRNAs-, cumate-, CymR- and/or cTA-dependent genetic switch(es); (d) modifications allowing accumulated PHB carbon to be made available for succinic acid production, e.g. cloned genes encoding PHB depolymerases and/or recycling enzymes; and (e) inhibition/inactivation of one or more gene(s) encoding succinate dehydrogenase paralogues and/or orthologues, e.g. genes encoding a L-aspartate oxidase and/or a succinate dehydrogenase flavoprotein subunit. For instance, the heterologous expression of one or more genes involved in succinic acid production, e.g. pyc, ppc, and/or icl, is achieved in a strain modified to allow accumulated PHB carbon to be made available for succinic acid production. In some embodiments, the bacterium as defined herein comprises heterologous expression of a polynucleotide encoding isocitrate lyase. In some embodiments, the bacterium as defined herein comprises overexpression of a protein involved in isocitrate synthesis (e.g., a citrate synthase, an aconitase, or both a citrate synthase and an aconitase). In some embodiments, the citrate synthase is gltA and/or said aconitase is acnA. In some embodiments, the overexpression of the protein involved in isocitrate synthesis is effected by expression of a heterologous polynucleotide encoding same.

In some embodiments, the bacterium as defined herein may be further modified to inhibit, reduce, or eliminate the activity of a protein involved in the Ethyl-Malonyl-CoA (EMC) pathway (e.g., by the knockout, knockdown, deletion or inactivation of a gene encoding said protein involved in the EMC pathway). In some embodiments, the protein involved in the EMC pathway is: (a) a protein that catalyzes the synthesis of acetoacetyl-CoA from acetyl-CoA; (b) a protein that catalyzes the synthesis of hydoxybutyl-CoA (OHB-CoA) from acetoacetyl-CoA; or (c) both (a) and (b). In some embodiments, the protein involved in the Ethyl-Malonyl-CoA (EMC) pathway is a beta-ketothiolase (e.g., PhaA), an acetoacetyl-CoA reductase (PhaB), an NADPH-linked acetoacetyl-CoA reductase, or any combination thereof.

According to another aspect, methods for preparing succinic acid or a salt thereof are described, the method comprising a step of growing a bacterium as herein defined in the presence of one or more C1-compound(s), for example a C1-compound comprising methanol or methane. In one embodiment, the method further comprises supplementation with malic acid or a salt thereof. In another embodiment, the bacterium is grown without additional supplementation with malic acid or a salt thereof. For instance, the bacterium is an sdh gap double mutant overexpressing a succinyl-CoA synthetase and is grown without additional supplementation with malic acid or a salt thereof during cultivation, e.g. malic acid being added only initially in the culture media.

In a further aspect, a method for preparing succinic acid is described, the method comprising a step of growing a C1-utilizing bacterium as herein defined in the presence of at least one C1-compound, wherein the activity of a TCA cycle succinate dehydrogenase (Sdh) is inhibited or reduced in said bacterium.

According to yet another aspect, a method for the preparation of a genetically engineered C1-utilizing bacterium is described, the method comprising a step of deleting at least one gene encoding an Sdh protein. In one embodiment, the method further comprises deleting one or more gene(s) encoding phasin(s), e.g. a gap gene. In another embodiment, the method further comprises overexpressing in the bacterium, a succinyl-CoA synthetase.

In some embodiments, the present description relates to one or more of the following items:
1. A genetically engineered C1-utilizing bacterium, wherein said bacterium is modified to disrupt a gene encoding a tricarboxylic acid (TCA) cycle succinate dehydrogenase (Sdh) or a subunit thereof.
2. The bacterium of item 1, wherein said bacterium is a serine cycle methylotroph bacterium.
3. The bacterium of item 2, wherein said serine cycle methylotroph bacterium is from the genera Burkholderia, Fulvimarina, Granulibacter, Hyphomicrobium, Methylibium, Methylobacterium, Ruegeria, preferably Methylobacterium.
4. The bacterium of item 1, wherein said bacterium is a serine cycle methanotroph bacterium.
5. The bacterium of item 4, wherein said serine cycle methanotroph bacterium is from the genera Methanomonas, Methylocystis, Methylocapsa, Methylocella, Methylococcus and Methylosinus, preferably Methylosinus.
6. The bacterium of any one of items 1 to 5, wherein said bacterium is modified by the knockout, knockdown or deletion of an sdh gene.
7. The bacterium of item 6, wherein said gene is an sdhA gene.
8. The bacterium of any one of items 1 to 7, wherein said bacterium is further modified to inhibit, reduce or eliminate the activity of a protein involved in polyhydroxyalkanoate biosynthesis and/or polyhydroxyalkanoate granule homeostasis.
9. The bacterium of item 8, wherein said bacterium is modified by the knockout, knockdown, deletion, or inactivation of a gene encoding said protein.
10. The bacterium of item 8 or 9, wherein said polyhydroxyalkanoate is poly-β-hydroxybutyric acid (PHB).
11. The bacterium of any one of items 8 to 10, wherein said protein involved in polyhydroxyalkanoate biosynthesis and/or polyhydroxyalkanoate granule homeostasis is a Granule-Associated Protein (GAP), a phasin, a PHB synthase, Gap11, Gap 20, PhaC, or PhaR.
12. The bacterium of any one of items 1 to 11, wherein said bacterium further comprises overexpression of a TCA cycle succinyl-CoA synthetase.
13. The bacterium of item 12, wherein the succinyl-CoA synthetase is SucC and/or SucD.
14. The bacterium of item 12 or 13, wherein said overexpression comprises an insertion of a P_{mxaF}sucCD DNA fragment into a chromosome.
15. The bacterium of any one of items 1 to 14, further comprising one or more of the following:
   (a) overexpression of one or more serine-cycle enzymes through modifications to their corresponding genes, for instance glyA, eno and/or mdh genes, encoding respectively serine hydroxymethyltransferase, enolase and malate dehydrogenase enzymes;
   (b) heterologous expression of one or more genes involved in succinic acid production, e.g. pyc (encoding a pyruvate carboxylase), ppc (encoding a phosphoenol pyruvate carboxylase), and/or icl (encoding isoctrate lyase);
   (c) incorporation of genetic switch(es), e.g. sRNAs-, cumate-, CymR- and/or cTA-dependent genetic switch(es);
   (d) modifications allowing accumulated PHB carbon to be made available for succinic acid production, e.g. cloned genes encoding PHB depolymerases and/or recycling enzymes; and
   (e) inhibition/inactivation of one or more gene(s) encoding succinate dehydrogenase paralogues and/or orthologues, e.g. genes encoding a L-aspartate oxidase and/or a succinate dehydrogenase flavoprotein subunit.
16. The bacterium of item 15, further comprising heterologous expression of a polynucleotide encoding isocitrate lyase.
17. The bacterium of any one of items 1 to 16, further comprising overexpression of a protein involved in isocitrate synthesis.
18. The bacterium of item 17, wherein said protein involved in isocitrate synthesis is a citrate synthase, an aconitase, or both a citrate synthase and an aconitase.
19. The bacterium of item 18, wherein said citrate synthase is gltA and/or said aconitase is acnA.
20. The bacterium of any one of items 17 to 19, wherein said overexpression of a protein involved in isocitrate synthesis is effected by expression of a heterologous polynucleotide encoding same.
21. The bacterium of any one of items 16 to 20, wherein said bacterium is further modified to inhibit, reduce, or eliminate the activity of a protein involved in the Ethyl-Malonyl-CoA (EMC) pathway.
22. The bacterium of item 21, wherein said bacterium is modified by the knockout, knockdown, deletion or inactivation of a gene encoding said protein involved in the EMC pathway.
23. The bacterium of item 21 or 22, wherein said protein involved in the EMC pathway is:
   (a) a protein that catalyzes the synthesis of acetoacetyl-CoA from acetyl-CoA;
   (b) a protein that catalyzes the synthesis of hydoxybutyl-CoA (OHB-CoA) from acetoacetyl-CoA; or
   (c) both (a) and (b).
24. The bacterium of item 23, wherein said protein involved in the Ethyl-Malonyl-CoA (EMC) pathway is a beta-ketothiolase, an acetoacetyl-CoA reductase, an NADPH-linked acetoacetyl-CoA reductase, or any combination thereof.
25. The bacterium of item 24, wherein: (i) said beta-ketothiolase is PhaA; (ii) said acetoacetyl-CoA reductase is PhaB, or both (i) and (ii).
26. A method for preparing succinic acid or a salt thereof, said method comprising a step of growing the bacterium as defined in any one of items 1 to 25 in the presence of one or more C1-compound(s).
27. The method of item 26, wherein said C1-compound comprises methane.
28. The method of item 26, wherein said C1-compound comprises methanol.
29. The method of any one of items 26 to 28, further comprising supplementation with malic acid or a salt thereof during cultivation.
30. The method of any one of items 26 to 29, wherein the bacterium is grown without additional supplementation with malic acid or a salt thereof during cultivation, other than malic acid added initially to the culture media.
31. A method for preparing succinic acid, said method comprising a step of growing a C1-utilizing bacterium in the presence of at least one C1-compound, wherein the activity of a TCA cycle succinate dehydrogenase (Sdh) is inhibited, reduced or eliminated in said bacterium.
32. The method of item 31, wherein said C1-compound is methanol.
33. The method of item 31 or 32, wherein said bacterium is a serine cycle methylotroph bacterium.
34. The method of item 33, wherein said serine cycle methylotroph bacterium is from the genera Burkholderia, Fulvimarina, Granulibacter, Hyphomicrobium, Methylibium, Methylobacterium, Ruegeria, preferably Methylobacterium.
35. The method of item 31, wherein said C1-compound is methane.
36. The method of item 31 or 35, wherein said bacterium is a serine cycle methanotroph bacterium.
37. The method of item 36, wherein said serine cycle methanotroph bacterium is from the genera Methanomonas, Methylocystis, Methylocapsa, Methylocella, Methylococcus and Methylosinus, preferably Methylosinus.
38. The method of any one of items 31 to 37, wherein said bacterium is modified by the knockout, knockdown or deletion of an sdh gene.
39. The method of item 38, wherein said gene is an sdhA gene.
40. The method of any one of items 31 to 39, wherein the activity of a protein involved in polyhydroxyalkanoate biosynthesis and/or polyhydroxyalkanoate granule homeostasis is inhibited, reduced or eliminated in said bacterium.
41. The method of item 40, wherein said bacterium is modified by the knockout, knockdown, deletion, or inactivation of a gene encoding said protein.
42. The method of item 40 or 41, wherein said polyhydroxyalkanoate is a poly-β-hydroxybutyric acid (PHB).
43. The method of any one of items 40 to 42, wherein said protein involved in polyhydroxyalkanoate biosynthesis and/or polyhydroxyalkanoate granule homeostasis is a Granule-Associated Protein (GAP), a phasin, a PHB synthase, Gap11, Gap 20, PhaC, or PhaR.
44. The method of any one of items 40 to 43, wherein said bacterium further comprises overexpression of a TCA cycle succinyl-CoA synthetase in the bacterium.
45. The method of item 44, wherein the succinyl-CoA synthetase is SucC and/or SucD.
46. The method of item 44 or 45, wherein said overexpression comprises an insertion of a P_{mxaF}sucCD DNA fragment into a chromosome.
47. The method of any one of items 31 to 46, wherein said bacteria further comprises one or more of the following:
   (a) overexpression of one or more serine-cycle enzymes through modifications to their corresponding genes, for instance glyA, eno and/or mdh genes, encoding respectively serine hydroxymethyltransferase, enolase and malate dehydrogenase enzymes;
   (b) heterologous expression of one or more genes involved in succinic acid production, e.g. pyc (encoding a pyruvate carboxylase), ppc (encoding a phosphoenol pyruvate carboxylase), and/or icl (encoding isoctrate lyase);
   (c) incorporation of genetic switch(es), e.g. sRNAs-, cumate-, CymR- and/or cTA-dependent genetic switch(es);
   (d) modifications allowing accumulated PHB carbon to be made available for succinic acid production, e.g. cloned genes encoding PHB depolymerases and/or recycling enzymes; and
   (e) inhibition/inactivation of one or more gene(s) encoding succinate dehydrogenase paralogues and/or orthologues, e.g. genes encoding a L-aspartate oxidase and/or a succinate dehydrogenase flavoprotein subunit
48. The method of any one of items 31 to 47, wherein said bacterium is as defined in any one of items 16 to 25.
49. The method of any one of items 31 to 48, wherein the bacterium is grown in the presence of malic acid supplementation.
50. The method of any one of items 31 to 48, wherein the bacterium is grown without additional malic acid supplementation.

Other features and advantages of the present invention will be better understood upon reading of the description herein below with reference to the appended drawings.

### DESCRIPTION OF THE DRAWINGS

FIG. 1 illustrates the methanol assimilation pathway of serine cycle methylotroph bacteria, including the methanol dissimilation pathway, the serine cycle, the Ethyl-Malonyl-CoA (EMC) pathway, the poly-β-hydroxybutyric acid (PHB) pathway, and the tricarboxylic acid (TCA) cycle. List of Abbreviations in FIG. 1: SER: serine; HPR: hydroxypyruvate; GLYC: glycerate; 2PG: 2-phospho-glycerate; PEP: phosphoenolpyruvate; OAA: oxaloacetate; MAL: malate; Ma-CoA: malyl-CoA; GLX: glyoxylate; GLY: glycine; Ac-CoA: acetyl-CoA; AcAc-CoA: acetoacetyl-CoA; MeMa-CoA: methylmalyl-CoA; P-CoA: propionyl-CoA; Suc-CoA: succinyl-CoA; SUC: succinate; FUM: fumarate; CIT: citrate; Iso-CIT: isocitrate; αKG: alpha-ketoglutarate; OHB-CoA: hydroxybutanoyl-CoA; PHB: poly-β-hydroxybutyrate; OHB: hydroxybutanoate; AcAc: acetoacetate.

FIG. 2 illustrates examples of modifications to the metabolic pathway of a serine-cycle methylotroph/methanotroph succinic acid producer strain. White triangles indicate the direction of the carbon flow toward succinic acid. Thickness of the pathway lines is proportional to the relative intensity of the carbon flux during methylotrophic growth. (1) The white dotted arrow marked by an “X” represents any genetic modifications resulting in reduced PHB accumulation or complete abolition of its synthesis. Examples, without limitation, include inactivation of gap20, phaC genes and/or overexpression of PHB depolymerases. (2) The grey arrow represents overexpression of any genes that pull the carbon flux toward succinic acid synthesis. An example, without limitation, includes the overexpression of the sucCD genes. (3) The white arrow marked with an “X” represents any mutation(s) within the sdh operon resulting in the inactivation of succinate dehydrogenase, i.e. loss of succinic acid oxidation activity and increase in succinic acid accumulation.

FIG. 3A is a graph showing succinic acid and malic acid concentrations as a function of growth (optical density) in a ΔsdhA mutant M. extorquens. FIG. 3B presents a graph showing growth (optical density) over time of a wild-type M. extorquens strain compared to its isogenic ΔsdhA mutant.

FIG. 4 shows comparative data for PHB production levels between wild-type M. extorquens, and its ΔsdhA, Δgap20, and ΔsdhA Δgap20 mutants.

FIGS. 5A-5B show malic acid and succinic acid concentrations as a function of optical density: (A) in the ΔsdhA Δgap20 double mutant cultured in 250 mL baffled Erlenmeyer flasks; and (B) in the ΔsdhA Δgap20 pCHOI2::sucCD strain cultured in 3-L baffled Erlenmeyer flasks.

FIGS. 6A-6C show malic acid and succinic acid concentrations as a function of optical density with the ΔsdhA Δgap20 Tn7::sucCD strain cultured: (A) in 250 mL baffled Erlenmeyer flasks with 1.5 g/L malic acid supplementation every 24 h, from day 3 till the end of experiment; (B) in 250 mL baffled Erlenmeyer flasks with addition of malic acid only at start; and (C) in 3-L baffled Erlenmeyer flasks with addition of malic acid only at start.

FIG. 7. Growth, succinic acid production, malic acid and methanol consumption in a ΔsdhA mutant of the wild-type strain M. extorquens ATCC55366. Methanol and malic acid were added only at the start of the experiment. The experiment was conducted using biological triplicates.

FIG. 8. Absolute succinic acid accumulation and yields obtained using different mutants of the wild-type strain M. extorquens ATCC55366 while supplementing with methanol during the course of the experiment. Malic acid was added only at the start of the experiment. (ODu: optical density unit). Experiments were conducted using biological triplicates.

FIG. 9. Succinic acid production and malic acid consumption in the ΔsdhA gap20 ΔphaC::Km^{R} triple mutant of the wild-type strain M. extorquens ATCC55366 while supplementing with methanol during the course of the experiment. This experiment is representative of two different experiments performed using 3 L baffled Erlenmeyer flasks. Malic acid was added only at the start of the experiment.

### SEQUENCE LISTING

The present application includes a sequence listing which lists the following sequences:

| SEQ ID | |
|---|---|
| NO: | Description |
| 1 | pCHOI2 vector nucleic acid sequence |
| 2 | Nucleic acid sequence of the sdh operon of Methylobacterium extorquens ATCC 55366, which |
| | includes: the sdhC gene from residues 942 to 1343; the sdhD gene from residues 1352 to 1771; the |
| | sdhA gene from residues 1801 to 3618; and the sdhB gene from residues 4280 to 5104 |
| 3 | Nucleic acid sequence comprising the gap20 gene from Methylobacterium extorquens ATCC |
| | 55366, which includes coding residues 501 to 941 |
| 4 | Nucleic acid sequence including the sucC (residues 1 to 1197) and sucD (residues 1206 to 2093) |
| | genes from Methylobacterium extorquens ATCC 55366 |
| 5 | the sdhA-up-F primer used in Example 1.1. |
| 6 | the sdhA-up-R primer used in Example 1.1. |
| 7 | the sdhA-down-F primer used in Example 1.1. |
| 8 | the sdhA-down-R primer used in Example 1.1. |
| 9 | the 5′-Forward primer of Example 1.3. |
| 10 | the 5′-Reverse primer of Example 1.3. |
| 11 | the sucC-BamHI-F primer of Example 1.4 |
| 12 | the sucD-Kpn1-R primer (Example 1.4). |
| 13 | the glmS-F primer of Example 1.4. |
| 14 | the dhaT-R primer of Example 1.4. |
| 15 | Eno-BamHI-F primer (Example 1.3) |
| 16 | Eno-Nhel-R primer (Example 1.3) |
| 17 | upPhaC-F primer (Example 1.5) |
| 18 | downPhaC-R primer (Example 1.5) |
| 19 | upPhaC-R primer (Example 1.5) |
| 20 | downPhaC-F primer (Example 1.5) |
| 21 | loxP-BamHI-F primer (Example 1.5) |
| 22 | loxP-BamHI-R primer (Example 1.5) |

### DETAILED DESCRIPTION

All technical and scientific terms used herein have the same meaning as commonly understood by one ordinary skilled in the art to which the invention pertains. For convenience, the meaning of certain terms and phrases used herein are provided below.

To the extent the definitions of terms in the publications, patents, and patent applications incorporated herein by reference are contrary to the definitions set forth in this specification, the definitions in this specification control. The section headings used herein are for organizational purposes only, and are not to be construed as limiting the subject matter disclosed.

The term “succinic acid” as used herein defines, 1,4-butanedioic acid, including its free acid or anionic forms like succinate salts.

The terms “C1”, “C1-compound”, “C1-carbon source” and equivalent expressions designate a molecule containing one carbon atom or containing two or more 1-carbon groups (e.g. methyl) not directly linked to each other. Examples of C1-compounds include, without limitation, methane, methanol, formaldehyde, formic acid, carbon monoxide, carbon dioxide, dimethyl ether, methyl formate, methylamine, dimethylamine, trimethylamine, and the like.

### C1-Utilizing Microorganisms

In some aspects, the present description relates to a C1-utilizing microorganism. More specifically, the present description relates to a C1-utilizing microorganism which is capable of accumulating a dicarboxylic acid (e.g., succinic acid) when growing on a C1-compound as a carbon source.

The expression “C1-utilizing” microorganism or similar expressions, as used herein, designates a microorganism like a bacteria or yeast, which assimilates and/or dissimilates C1-compounds as above-defined, and/or uses C1-compounds as carbon sources. These include, for example, methylotroph and methanotroph microorganisms.

In some embodiments, the C1-utilizing microorganism may be a methylotroph or a methanotroph. As used herein, the term “methylotroph” defines a group of microorganisms that can use C1-compounds, such as methanol, as the carbon source for their growth. Examples of methylotrophs include, without limitation, bacteria within the genera Burkholderia, Fulvimarina, Granulibacter, Hyphomicrobium, Methylibium, Methylobacterium, Ruegeria. In contrast, the terms “methanotroph” or “methanophile” define a group of microorganisms able to metabolize methane as their source of carbon. Methanotrophs include type I methanotrophs which use the ribulose monophosphate (RuMP) pathway, and type II methanotrophs which use the serine pathway for carbon assimilation. Examples of type I methanotrophs include, without limitation, bacteria within the genera Methylobacillus, Methylobacter, Methylococcus, Methylomonas, Methylophaga, Methylotenera, Methylophilales. Examples of type II methanotrophs include, without limitation, bacteria within the genera Methanomonas, Methylocapsa, Methylocella, Methylocystis and Methylosinus.

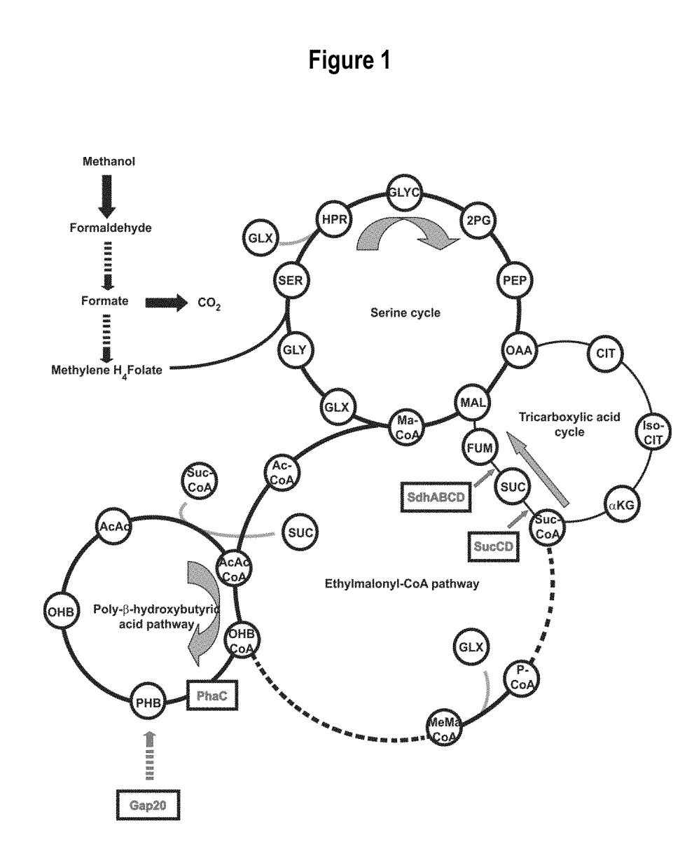

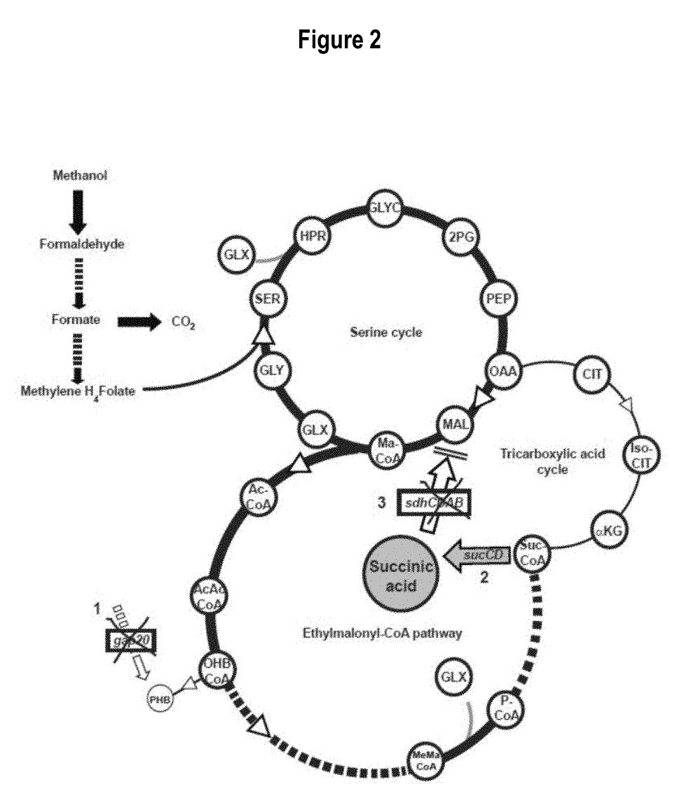

In some embodiments, the C1-utilizing microorganism may be a serine-cycle C1-utilizing microorganism. Serine cycle methylotrophs have the ability to consume methanol for their growth, and can therefore convert methanol to succinic acid through their one-carbon metabolism and tricarboxylic acid (TCA) cycles. Although the methanol assimilation pathway of a serine-cycle methylotrophic bacteria is illustrated in FIG. 1, other C1-utilizing microorganisms such as type II methanotrophic bacteria typically possess the same or substantially the same pathways, with the exception that they further include additional enzymatic step(s) achieving the transformation of methane into methanol (e.g., a methane monooxygenase (MMO)). As such, although the present description has been exemplified using a serine-cycle methylotrophic bacterium (Methylobacterium extorquens) in the present Examples, it is understood that the present teachings may extend to other C1-utilizing microorganisms such as methanotrophic bacteria⁷.

As an example, Methylosinus trichosporium, a serine cycle methanotroph has been intensively studied⁷ for its capacity to use methane as the sole source of carbon and energy, and could be modified as herein described and used to produce succinate from methane. Furthermore, this bacterium has also been recently used as a biocatalyst for the oxidation of methane to methanol⁸.

Methylobacterium extorquens is also a suitable C1-utilizing model in the present bioprocess to produce succinic acid. M. extorquens is a pink pigmented, non-pathogenic, Gram-negative serine-cycle methylotroph bacterium ubiquitous in the environment, and particularly associated with plants^{4,5}. M. extorquens can also be grown to very high cell densities using a controlled methanol supplied bioprocess²³.

M. extorquens' genes involved in methanol dissimilation and assimilation have been extensively studied since the 1960s^{6,9,10-21}. The dissimilation of methanol begins in the periplasm by its oxidation, forming formaldehyde (see FIG. 1). This reaction is catalysed by the methanol dehydrogenase (MDH) MxaFI, which carries a pyrroloquinoline quinone (PQQ) as prosthetic group and uses calcium as co-factor. The released electron is captured by the oxidized cytochome C and transferred to the electron transport chain, generating ATP. Then, formaldehyde is detoxified to formate within the cytoplasm through multiple enzymatic steps that uses the methanopterin tetrahydrofolate co-factor as electron carrier. Next, formate is dissimilated into CO_{2,} in a process using NAD⁺ as proton acceptor, or converted into methylene tetrahydrofolate.

Condensation of methylene-H₄F with glycine and water produces serine, thereby beginning the serine cycle. Acetyl-CoA supplied by the serine cycle is a branching point molecule with the Ethyl-Malonyl-CoA (EMC) pathway and poly-β-hydroxybutyrate (PHB) cycles. As depicted in FIG. 1, the EMC pathway involves successive thio-ester-CoA molecule modifications and flows into the TCA cycle by forming succinyl-CoA. Most importantly, the enzymatic reaction carried by the EMC enzyme MclA not only forms propionyl-CoA but also the glyoxylate required for assimilation of methanol. For instance, glyoxylate produces glycine through transamination, which, in turn, is involved in the first step of the serine cycle. Also, glyoxylate is implicated in the formation of hydroxypyruvate (HPR) within the serine cycle (FIG. 1).

The EMC pathway also shares its two first steps with the PHB cycle—i.e., the successive synthesis of acetoacetyl-CoA and hydoxybutyryl-CoA (OHB-CoA) from acetyl-CoA, achieved by PhaA, a β-ketothiolase, and PhaB, a NADPH-linked acetoacetyl-CoA reductase, respectively. The final step of PHB synthesis is performed by the PHB synthase PhaC. The genes depA, depB, hbd and atoAD are responsible for its depolymerisation into acetoacetyl-CoA. PHB belongs to the polyester family of polyhydroxyalkanoate (PHA) and is synthesized by M. extorquens and some other bacteria during nutrient and oxygen limitation²².

PHB producing bacteria such as M. extorquens accumulate PHB in their cytoplasm as granules which can account easily for 40% of the dry biomass²³⁻²⁵. Moreover, Granule-Associated Proteins (GAP) are important players of PHB granules homeostasis. Among GAPs, phasins are implicated in the regulation of granule size, stability, localization, number, and their segregation during cell division^{22,26,27}. Although their mechanisms of action are not fully understood, it has been shown that some phasins bind PHB synthases and depolymerases²⁸⁻³⁰. Other regulators such as PhaR, which controls acetyl-CoA flux and PHB synthesis, could also be associated to phasins in M. extorquens^{25,31}. For instance, at least two phasins have been identified in M. extorquens: Gap11 and Gap20^{24,25,32}.

Examples of challenges faced when producing succinic acid in C1-utilizing serine-cycle microorganisms, include the following: (i) the genes from the TCA cycle are poorly expressed during growth on methanol; (ii) an inactivating mutation within the TCA cycle was found lethal to the bacteria when grown on methanol as the sole carbon source; and (iii) PHB accumulated during growth on methanol. For instance, the bacterial strains and/or methods herein described were found to solve one or more of these issues as explained in more detail below.

(i) M. extorquens can use simultaneously both methanol and succinic acid for growth but the latter is preferred and more rapidly consumed than methanol¹⁴. Consequently, methanol may not be assimilated efficiently in sdh null mutants or sdh knockdown backgrounds, considering regulatory effects of succinic acid accumulation on TCA and EMC gene expression. Indeed, genes belonging to the TCA are poorly expressed during methylotrophic growth, with a noticeably weak aconitase (Acn) activity, reducing the oxidative TCA flux from citrate. Thus, in contrast to what is observed during growth on succinate, the TCA cycle is expressed at a weak basal level while the EMC is up-regulated during growth on methanol, thereby favoring methanol assimilation¹⁶. Similarly, feedback inhibition could also occur, thus down-regulating genes needed for succinic acid production. Nevertheless, as presented in Example 3.1, the inactivation of an sdh gene was sufficient to allow succinic acid accumulation in this bacterium when grown on a C1-compound.

(ii) Some bacterial species, such as Escherichia coli, can produce succinic acid as an electron sink, in rich media, when shifting from aerobic to anaerobic conditions³³. However, as M. extorquens is a strictly aerobic microbe. As such, one way of enhancing succinic acid production would be through metabolic engineering in the TCA cycle, for instance, by blocking the enzymatic conversion of succinate to fumarate. Unfortunately, an inactivating mutation within the succinate dehydrogenase operon sdhCDAhB, responsible for this step, is lethal when grown on methanol alone because it interrupts the TCA and thus, glyoxylate regeneration achieved by the EMC.

The TCA enzymes succinyl-CoA synthetase SucCD, succinate dehydrogenase SdhCDAB, and fumarate dehydrogenase FumC, complete the EMC flux¹⁰ and this allows for the formation of two molecules of glyoxylate per round of EMC and serine cycles. The TCA cycle supplements the serine cycle with malate, which is also essential for central metabolism. However, as shown in Example 3, succinic acid accumulation is possible with the sdh operon mutants if the growth media is supplemented with malate, which complements the incomplete TCA cycle.

(iii) M. extorquens accumulates PHB during growth on methanol and growth to high density obviously creates a nutrient limited environment also in favor of PHB synthesis²³⁻²⁵. As described in Example 4, succinic acid production by M. extorquens, using methanol as the source of carbon and energy, is further improved by modulating PHB reserves to promote succinic acid accumulation. In fact, the sdhA gap20 double mutant produced 4.76 fold less PHB than the ΔsdhA mutant.

### Genetic Modifications

While naturally-occurring C1-utilizing microorganisms have the ability to produce succinic acid as a TCA cycle metabolite, they generally do not accumulate significant amounts of succinic acid when grown on methanol. In fact, no accumulation of succinic acid was detected when the wild-type strain of the methylotrophic bacterium M. extorquens was cultured using methanol as the carbon source (Example 3.1). Accordingly, in some aspects, the present description relates to a C1-utilizing bacterium that has been genetically engineered to accumulate succinic acid (e.g., via the oxidative TCA pathway).

As used herein, the expression “modified”, “genetically modified”, “genetically engineered” or similar expressions associated with term microorganism or bacterium, refer to a microorganism or bacterium whose genome has been modified, for instance, by the addition, substitution and/or deletion of genetic material. Methods for modifying organisms are known and include, without limitation, random mutagenesis, point mutations, including insertions, deletions and substitutions, knockouts, transformations using recombinant nucleic acid sequences, including both stable and transient transformants.

Accordingly, in some aspects, the present description relates to a genetically engineered C1-utilizing bacterium that has been modified to disrupt a gene encoding a TCA cycle succinate dehydrogenase (Sdh) or a subunit thereof, thereby accumulating succinic acid from the oxidative TCA pathway. In some embodiments, the gene encoding the TCA cycle succinate dehydrogenase may be sdhA, sdhB, sdhC, sdhD, or any combination thereof.

As used herein, the expression “gene disruption” and equivalent expressions designate a genetic alteration that renders the encoded gene product inactive. The genetic alteration can be, for example, deletion of the entire gene, deletion of a regulatory sequence required for transcription or translation, deletion of a portion of the gene which results in a truncated gene product, or by any other mutation which inactivates the encoded gene product, for example via knockout or knockdown of the gene, or via one or more amino acid substitutions or deletions at residues critical for activity of the encoded protein. In some embodiments, where one or more genes are to be disrupted in accordance with the present description, one or more small RNAs (sRNAs) may be used to knockdown their expression. In some embodiments, a modified CRISPR system may also be used in a very similar way, for example using a catalytically inactive CRISPR endonuclease (e.g., a catalytically inactive Cas9).

In addition to the disruption of an sdh gene, the genetically engineered C1-utilizing microorganisms of the present description may be further modified, for example, to improve one or more of the following aspects: increasing succinic acid production, reducing PHB production or rendering PHB available as a carbon source for succinic acid production, and/or decreasing the need for malate supplementation.

PHB formation/accumulation can be reduced, for example, by blocking or reducing PHB synthesis directly, or by over-expressing PHB depolymerases. For instance, phasins are GAPs (granules-associated proteins) implicated in the regulation of granule size, stability, localization, number and their segregation during cell division. As such, inactivation (e.g., by gene deletion, knockout or knockdown) of one or more phasins such as gap11 or gap20 in M. extorquens, reduces PHB production during growth on methanol.

Other proteins involved in the PHB pathway could also be modulated. For instance, an sdhA phaC double mutant, that produces no PHB, but grows normally on methanol, could be obtained. On the other hand, modifications within the PHB pathway could also allow biomass accumulated in the form of PHB to be converted to succinic acid. For instance, this could be achieved by cloning genes encoding PHB depolymerases and recycling enzymes, alone or in combination, under an inducible promoter (see also Example 8).

Accordingly, in some embodiments, the genetically engineered C1-utilizing microorganism may further be modified to inhibit, reduce or eliminate the activity of a protein such as Granule-Associated Protein (GAP), a phasin, a PHB synthase, Gap11, Gap 20, PhaC, PhaR, or any combination thereof.

In some embodiments, the genetically engineered C1-utilizing microorganism may further be modified to overexpress PHB depolymerases and/or PHB recycling enzymes. In some embodiments, the genetically engineered C1-utilizing microorganism may further be modified to overexpress the gene depA, depB, hbd, atoAD, or any combination thereof, which are responsible for PHB depolymerisation into aceto-acetyl-CoA.

As used herein, the term “overexpression” and equivalent terms indicate that a particular gene product is produced at higher levels in a modified microorganism compared to its unmodified version. For example, a microorganism that includes a recombinant nucleic acid configured to overexpress an enzyme produces the enzyme at a greater amount than a microorganism that does not include the recombinant nucleic acid. The term “overexpression” when associated with a gene means an increased expression of such gene in a modified microorganism compared to its unmodified version. Gene overexpression, for instance, also results in the overexpression of its encoded gene product. Overexpression may be done by any means known in the art, such as by integration of additional copies of the target gene in the cell's genome, expression of the gene from an episomal expression vector, introduction of an episomal expression vector which comprises multiple copies of the gene, or by the use of a promoter heterologous to the coding sequence to which it is operably linked, i.e. the sequence coding for the gene product to be overexpressed.

Enzymes upstream of the Sdh protein in the TCA cycle may also be overexpressed through genetic modifications in order to improve succinic acid production and/or reduce the need for malate supplementation, preferably an enzyme common to both the TCA cycle and EMC pathway, e.g., overexpression of a succinyl-CoA synthetase.

Accordingly, in some embodiments, the genetically engineered C1-utilizing microorganism may further be modified to overexpress of a succinyl-CoA synthetase (e.g., a TCA cycle succinyl-CoA synthetase). In some embodiments, the succinyl-CoA synthetase may be SucC and/or SucD. In some embodiments, the succinyl-CoA synthetase may be inserted into the genome of the C1-utilizing microorganism (e.g., using a strong promoter such as the mxaF promoter).

Based on transcriptomic analysis, some genes belonging to the methanol dissimilation/assimilation pathway were found to be up-regulated (mtdA, fch and most serine cycle genes) in the sdhA mutant model. Without wishing to be bound by theory, it can be deduced that the sdhA mutation acts in synergy with methanol and further increases expression of methanol assimilation genes.

The transcriptomic analysis showed that gck and mtk expression was up-regulated, whereas eno and mdh genes were not differentially expressed, when comparing the sdhA mutant to the wild-type ATCC55366 strain (see Example 3.2). Overexpression of proteins encoded by the glyA (serine hydroxymethyltransferase), eno (enolase), and mdh (malate dehydrogenase enzyme) genes within the sdhA mutant is expected to promote the continuous flow of the serine cycle as well as the synthesis of acetyl-CoA.

Accordingly, in some embodiments, the genetically engineered C1-utilizing microorganism may further be modified to overexpress a serine hydroxymethyltransferase, an enolase, a malate dehydrogenase, or any combination thereof.

Some succinate dehydrogenase activity may still be present within the modified strain, e.g. through sdh paralogues and/or orthologues. If it would be the case, succinic acid accumulation would be slowed down and eventually consumption would overtake synthesis. As such, one or more genes encoding sdh paralogues and/or orthologues may also be inactivated. Thus, in some embodiments, the genetically engineered C1-utilizing microorganism may further be modified to disrupt sdh paralogues and/or orthologues. In some embodiments, the genetically engineered C1-utilizing microorganism may be further modified to disrupt an L-aspartate oxidase and/or a succinate dehydrogenase flavoprotein subunit.

In some embodiments, the genetically engineered C1-utilizing microorganism (e.g., an sdhA mutant) may also be complemented using genetic switches, such as described in Example 9. Such switches may be employed for example to eliminate the need for initial malate addition for growth on methanol to produce succinic acid, by controlling the expression of a TCA cycle succinate dehydrogenase (Sdh) or a subunit thereof (e.g., an sdh operon). Sdh proteins produced from such switches are expected to be exhausted later on during growth and succinic acid would then accumulate. In some embodiments, the genetic switch may be a cumate-dependent genetic switch. In some embodiments, the genetically engineered C1-utilizing microorganism may comprise one or more genetic switch(es) such as sRNAs-, cumate-, CymR- and/or cTA-dependent genetic switch(es).

In some embodiments, the genetically engineered C1-utilizing microorganism may also be further modified through heterologous gene expression. More specifically, in some embodiments, the genetically engineered C1-utilizing microorganism may be further modified to overexpress enzymes responsible for the conversion of pyruvate and PEP into OAA. In some embodiments, such enzymes may be a pyruvate carboxylase (e.g., encoded by the pyc gene) and/or a phosphoenolpyruvate (PEP) carboxylase (e.g., encoded by the ppc gene). The overexpression of such proteins has been shown to improve aerobic succinate production in some bacteria^{54,55}. The increase of the OAA pool within M. extorquens cells is expected to provide more carbon input into the EMC, especially if the mdh gene is also functionally overexpressed.

In some embodiments, the above mentioned pyc gene may be from Rhodopseudomonas palustris BisA53, which is an environmental non-pathogenic bacteria belonging to the rhizobiale group of alphaproteobacteria⁵⁶.

In some embodiments, the genetically engineered C1-utilizing microorganism may also be further modified to overexpress an enzyme that catalyzes the formation of glyoxylate and succinate from isocitrate (e.g., an isocitrate lyase)^{57,58}. Isocitrate lyase is a key enzyme of the glyoxylate regeneration pathway and is absent from the M. extorquens genome, which uses the EMC pathway. For example, isocitrate lyase may be used to increase the oxidative flux from citrate within the TCA, which may occur as succinic acid accumulates in a genetic switch complemented sdhA mutant. Heterologous overexpression of an isocitrate lyase within a genetically engineered C1-utilizing microorganism of the present description could also allow subsequent inactivation of the EMC pathway, which theoretically would result in a larger amount of carbon available for succinic acid production. This would involve introducing a heterologous glyoxylate shunt, as described in more detail in Example 13. Briefly, isocitrate produced by the TCA cycle can be converted by the heterologous isocitrate lyase to form glyoxylate and succinate, instead of the isocitrate being further decarboxylated (by isocitrate dehydrogenase). The glyoxylate can then be used together with acetyl-CoA to produce malate (e.g., by malate synthase), making the missing carbon to enter the central metabolism (and thus potentially reducing the need for malate).

Accordingly, in some embodiments, the genetically engineered C1-utilizing microorganism (e.g., expressing heterologous isocitrate lyase) may also be further modified to overexpress of a protein involved in isocitrate synthesis (e.g., a citrate synthase (e.g., gltA), an aconitase (e.g., acnA), or both a citrate synthase and an aconitase). In some embodiments, the genetically engineered C1-utilizing microorganism (e.g., expressing heterologous isocitrate lyase) may also be further modified to overexpress a malate synthase, and/or to disrupt a gene encoding an isocitrate dehydrogenase.

In some embodiments, the genetically engineered C1-utilizing microorganism (e.g., expressing heterologous isocitrate lyase) may also be further modified to inhibit, reduce, or eliminate the activity of a protein involved in the EMC pathway. In some embodiments, the protein involved in the EMC pathway may be: (a) a protein that catalyzes the synthesis of acetoacetyl-CoA from acetyl-CoA; (b) a protein that catalyzes the synthesis of hydoxybutyryl-CoA (OHB-CoA) from acetoacetyl-CoA; or (c) both (a) and (b). In some embodiments, the protein involved in the EMC pathway may be a beta-ketothiolase, an acetoacetyl-CoA reductase, an NADPH-linked acetoacetyl-CoA reductase, or any combination thereof. In some embodiments, (i) the beta-ketothiolase may be PhaA; (ii) the acetoacetyl-CoA reductase may be PhaB; or both (i) and (ii).

In some embodiments, where one or more genes are to be disrupted in accordance with the present description, one or more small RNAs (sRNAs) may be used to knockdown their expression, as described in Example 12. In some embodiments, a modified CRISPR system may also be used in a very similar way, for example using a catalytically inactive CRISPR endonuclease (e.g., a catalytically inactive Cas9).

### Methods

In some aspects, the present description relates to a method for preparing succinic acid or a salt thereof. The method generally comprises growing a genetically engineered C1-utilizing microorganism as defined herein in the presence of one or more C1-compound(s). In some embodiments, the C1-compound may comprise methane and/or methanol.

In some embodiments, the method may comprise supplementing the culture with malic acid or a salt thereof. In some embodiments, the genetically engineered C1-utilizing microorganism may be grown without additional supplementation with malic acid or a salt thereof during cultivation, other than malic acid added initially to the culture media. In some embodiments, the genetically engineered C1-utilizing microorganism may be grown without the addition malate during culture, or may require less malate during culture (e.g., for genetically engineered C1-utilizing microorganisms comprising genetic switches to control TCA cycle metabolism, and/or for genetically engineered C1-utilizing microorganisms comprising an operative glyoxylate shunt pathway).

### EXAMPLES

The following examples are for illustrative purposes and should not be construed as further limiting the invention as herein described.
Bacterial strains, plasmids, media and cultures: Bacterial strains and plasmids are listed in Table 1. Escherichia coli strains were grown using Tryptic Soy Broth (TSB) and Agar (TSA). Methylobacterium extorquens was grown using the CHOI4 medium (see Table 2), which was developed to yield high cell density fermentation²³. M. extorquens cultures were carried out using 250 or 3000 mL baffled Erlenmeyer flasks containing 35 or 300 mL of CHOI4 medium, respectively. Strains were grown at 30° C., 250 rpm and cultures were supplemented initially with 0.5% methanol and 0.5% every 24 h, unless indicated otherwise. Malic acid was added as indicated. Each time point corresponds to 24 h. Antibiotics were used at the following final concentrations: carbenicillin, 100 μg/mL; kanamycin, 40 μg/mL; tetracyclin, 10 μg/mL; and streptomycin, 50 μg/mL.

TABLE 1
Bacterial strains and plasmids

| | | |
|---|---|---|
| Bacterial strain | | |
| or plasmid | Relevant characteristics | Reference or source |

Escherichia coli strains

| | | |
|---|---|---|
| SM10λpir | thi-1, leu, tonA, lacY, supE, recA::RP4-2-Tc::Mu, λpir, Km^{r} | Miller and Mekalanos, |
| | | 1988, J. Bacteriol |
| | | 170:6, 2575-83 |
| χ7213 | SM10λpir Δasd | Reference 35 |

Methylobacterium extorquens strains

| | | |
|---|---|---|
| ATCC55366 | Wild-type | Bourque et al, 1992, |
| | | App Microbiol |
| | | Biotechnol, 37:7-12 |
| ΔsdhA | Polar mutation, marker less | See Example 1.1 |
| gap20 | May also be referred to as “gap20::145”, 145 bp insertion (scar | See Example 4 |
| | from Cre-Lox), Marker less | |
| ΔsdhA gap20 | Derived from ΔsdhA and gap20 strains, marker less | See Example 4 |
| ΔsdhA gap20 ΔphaC | Derived from the ΔsdhA gap20 strain, ΔphaC refers to the | See Example 5 |
| | complete deletion of phaC together with the 5′ end of its | |
| | upstream gene, marker less, PHB negative | |
| ΔsdhA gap20 | Triple mutant described above before the Cre-Lox | See Example 7 |
| ΔphaC::Km^{R} | recombination, Km^{r}, PHB negative, with chromosomal | |
| Tn7::sucCD | insertion of sucCD genes under the methanol dehydrogenase | |
| | promoter P _{mxaF}, Tet^{r} | |
| ΔsdhA gap20 ΔphaC | The marker less triple mutant described above overexpressing | See Example 8 |
| pCHOI2::eno (Km^{R}) | the eno gene onto a plasmid (see below) | |

Plasmids

| | | |
|---|---|---|
| pGEM-T easy | TA cloning vector | Promega Madison, WI, |
| | | USA |
| pCHOI2::sucCD | pCM110 derived, P_{mxaF}, Tet^{r} | Reference 36, and |
| | | Example 1.4 |
| pUC18T mini- | FRT, Tet^{r} | Reference 39, and |
| Tn7T::P_{mxaF}sucCD | | Example 1.4 |
| pCHOI2::eno (Km^{R}) | P_{mxaF}, Tet^{r} | Examples 1.3 and 8 |

Km^{r}: Kanamycin;
Tet^{r}: Tetracycline

TABLE 2
CHOI4 Medium for M. extorquens high cell density fermentation

| | Chemicals | Final concentration (mM) |
|---|---|---|
| Solution | (NH₄)₂SO₄ | 11.35 |
| 1 | KH₂PO₄ | 9.57 |
| | Na₂HPO₄ | 15 |
| | MgSO₄ | 5.48 |
| | | Final concentration (μM) |
| Solution | MnSO₄ | 87 |
| 2 | ZnSO₄ | 27 |
| | CuSO₄ | 10 |
| | Na₂MoO₄ | 10 |
| | CoCl₂ | 10 |
| | H₃BO₃ | 29 |
| | FeSO₄ | 216 |
| | CaCl₂ | 408 |

### Example 1

### Construction of Mutant Strains and Vectors

1.1—Construction of the ΔsdhA Mutant

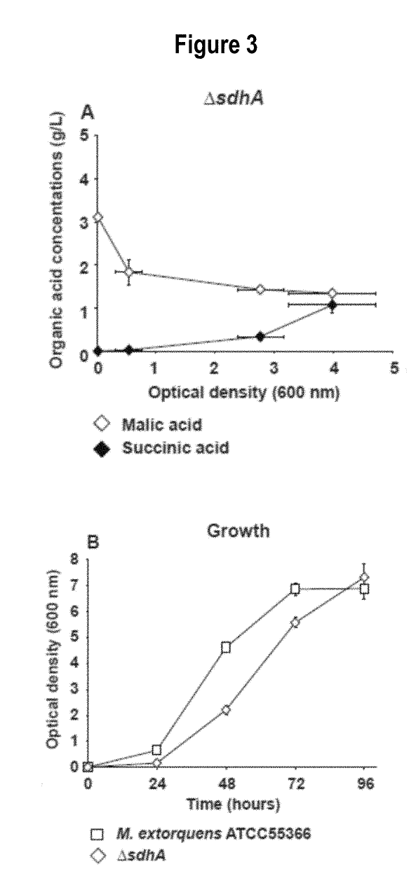

In general terms, the sdhA gene is deleted using the pCM184 allelic exchange vector technology. This technology is described in FIG. 3 of Marx & Lidstrom (reference 34). Briefly, the loxP-Km-loxP portion of the vector is inserted within the genome to replace the sdhA gene. The kanamycin marker (Km) is then removed from the mutants using pCM157, leaving only loxP. Positive clones (with the gene deletion) are then selected and the ΔsdhA mutation confirmed by sequencing.

More specifically, Phusion™ High fidelity DNA polymerase (New England BioLabs, Inc., Ipswich, Mass., USA) was used for all DNA amplifications. All restriction enzymes used herein were from NEB as well. Linear fragments were circularized using the T4 DNA ligase from NEB. Genomic regions located upstream and downstream of the M. extorquens ATCC55366 sdhA gene were amplified using the following two primer pairs:

sdhA-up-F:
(SEQ ID NO: 5)
5′-GAATTCCTGATGCTCGCCTTCGTC-3′/
sdhA-up-R:
(SEQ ID NO: 6)
5′-GCGGCCGCTGCTCGAGTTCGTA GAC-3′,
containing the EcoRI and NotI restriction sites
respectively (underlined);
and
sdhA-down-F:
(SEQ ID NO: 7)
5′-GGGCCCGTCGTGACCATGGAATC-3′/
sdhA-down-R:
(SEQ ID NO: 8)
5′-GAGCTCGCTGCCGCGGTAGA-3′,
containing the ApaI and SacI restriction sites
respectively.

Each fragment was cloned into the TA cloning vector pCRII (Life Technologies). The E. coli DH5α strain (Life Technologies) was used for propagation. Then, each fragment was excised from pCRII using the corresponding restriction enzymes and successively cloned into the allelic exchange vector pCM184³⁴. The resulting pCM184::ΔsdhA-loxP-Km-loxP-ΔsdhA vector was mobilized into M. extorquens recipient strains using the Δasd Sm10λpir strain χ7213³⁵. On-filter conjugation was allowed to occur during 16 h at 37° C. on Luria plates containing diaminopimelate (DAP). Filters were transferred onto CHOI4 agar plates and incubated at 30° C. for 24 hours.

Growing clones were then diluted in PBS and different volumes were spread out on CHOI4 agar plates containing kanamycin. The kanamycin marker was removed from the ΔsdhA mutants using the cre-lox system³⁴. Mutants were transformed with the Cre recombinase positive vector pCM157 and grown in CHOI4 medium containing tetracycline. Then, kanamycin negative clones were selected and further grown in CHOI4 medium without any antibiotic selective pressure, to promote the loss of pCM157. Kanamycin and tetracycline negative clones were screened by PCR for the marker less ΔsdhA mutation, using the sdhA-up-F and sdhA-down-R primers. A positive clone was selected and the ΔsdhA mutation was confirmed by sequencing.

1.2—Construction of the ΔsdhA gap20 Double Mutant

A 910 bp fragment containing gap20 and its flanking regions was amplified by PCR and cloned into the pCRII vector, giving pCRII::gap20. The gentamycin resistance marker (Gm) together with its loxP flanking sites was amplified from pCM351³⁴ using primers containing either HincII or BpII restriction site. The resulting fragment was cut with HincII and BpII and cloned into pCRII::gap20 linearized using the same enzymes, giving pCRII:Δgap20Gm^{r}. The Δgap20Gm^{r} fragment was amplified by PCR and used to transform by electroporation the marker ΔsdhA mutant strain from Example 1.1. Clones were selected on CHOI4 agar plates containing gentamycin. The gentamycin marker was removed from the ΔsdhA gap20 double mutant using the cre-lox system as described above³⁴.

1.3—Construction of the pCHOI2 Vector and pCHOI2::eno

The pCHOI2 vector was constructed from the pCM110 vector³⁶. Km resistance gene was amplified using the pNEW vector³⁷ as a template with primers 5′-Forward-CTGCAGATGATTGAACAAGATGG-3′ (SEQ ID NO: 9) and 5′-Reverse-CTGCAGTCAGAAGAACTCGTCAAGAA-3′ (SEQ ID NO: 10), each containing the PstI restriction site in 5′. PCR product was introduced into pCM110 digested with PstI and the positive colonies were selected on plates containing kanamycin. Then, tetA and tetR genes were removed by double digestion with AfeI and FspI. MCS from pSL1190³⁸ (Genbank accession #U13866) was introduced into the blunt ended vector to complete pCHOI2.

The eno gene was amplified using the following primers:

Eno-BamHI-F:
(SEQ ID NO: 15)
AAAAAA-GGATCC-ATGACCGCGATCACCAATATC
and
Eno-NheI-R:
(SEQ ID NO: 16)
AAAAAA-GCTAGC-atgcttcaggtgcgaTCAGC,

giving a PCR fragment of 1305 bp. The fragment was cut with BamHI and NheI and cloned into pCHOI2 cut with the same enzymes and propagated in E. coli DH5α. The resulting pCHOI2::eno was introduced in M. extorquens strains by electroporation using a Biorad apparatus (2.5 Kv, 200Ω).
1.4—Construction of pCHOI2::sucCD and Tn7::sucCD

The M. extorquens ATCC55366 sucCD genes were amplified using the primers sucC-BamHI-F: 5′-GGATCCATGAACATCCACGAATACCA-3′ (SEQ ID NO: 11) and sucD-Kpn1-R 5′-GGTACCTCACCTGGACTTCAGCAC-3′ (SEQ ID NO: 12). The resulting PCR fragment was cloned into the TA cloning vector pGEM-T easy (Promega) and propagated in E. coli DH5α. The sucCD genes were then excised using BamIH and SacI and introduced downstream of the mxaF promoter (P_{mxaF}), in the pCHOI2 vector linearized with the same enzymes. The resulting pCHOI2::sucCD was introduced in M. extorquens strains by electroporation using a Biorad apparatus (2.5 Kv, 200Ω).

For chromosome insertion, a modified version of pUC18T-miniTn7T-Gm³⁹, carrying a tetracycline marker within SacI of the MCS, was used. Briefly, the P_{mxaF}sucCD fragment was excised from the pCHOI2::sucCD vector using the HindIII and KpnI restriction enzymes and introduced in the pUC18T-miniTn7T vector. Conjugation was performed as described above and clones were selected on CHOI4 agar plates containing tetracycline.

Insertion of the Tn7 into the glmS-dhaT integration site was confirmed by PCR using the glmS-F: 5′-CGAGAAGACTGTCTCGAAC-3′ (SEQ ID NO: 13) and dhaT-R: 5′-CATCGCGATTGTCGATTCG-3′ (SEQ ID NO: 14) primers. Integration occurs within a noncoding region of the chromosome, making the insert stable and silent in regard of the surrounding genes⁴⁰.
Antibiotic markers are removed from the different genetic constructs using Cre-Lox or flipase technologies, making the final M. extorquens engineered strain suitable for bioprocesses purpose³⁴.
   1.5—Construction of the ΔsdhA gap20 ΔphaC Triple Mutant

A 3719 bp fragment containing phaC and its flanking regions was amplified by PCR using the following primers: upPhaC-F: 5′-ATGTTGGCGAAGCCCTCCTTC-3′ (SEQ ID NO: 17) and downPhaC-R: 5′-GATTCGGCGAGCACCATTCC-3′ (SEQ ID NO: 18). The resulting fragment was cloned into the pGEM-T easy vector (Promega), giving pGEM-T easy::phaC. Then, the phaC gene was deleted by performing an inverse PCR using the following BamHI containing primers: upPhaC-R: 5′-GGATCCACACGTCCTCCCAAAGGT-3′ (SEQ ID NO: 19) and downPhaC-F: 5′-GGATCCTGAAGGTGTGAGGGATCG-3′ (SEQ ID NO: 20); giving the linear pGEM-T easy::Δphac fragment.

A 1340 bp fragment containing a kanamycin resistance cassette flanked on both sides by the loxP recombination recognition sequence was amplified from pCM184 (Marx and Lidstrom, 2002) using the following BamHI containing primers: loxP-BamHI-F: 5′-GGATCCGCATAACTTCGTATAGCATAC-3′ (SEQ ID NO: 21) and loxP-BamHI-R: 5′-GATAAGCTGGATCCATAACTTCG-3′ (SEQ ID NO: 22); giving the loxP-Km^{R}-loxP fragment. The pGEM-T easy::ΔphaC fragment cut with BamHI was then resolved with the loxP-Km^{R}-loxP fragment cut with the same enzyme. The ΔphaC::Km^{R} fragment was finally cloned into the suicide vector pCM433 (Marx, 2008). Conjugation was performed as described for the ΔsdhA mutant, using the ΔsdhA gap20 double mutant as recipient strain. Then, to select the double-crossover allele replacement, a kanamycin resistant clone was grown in CHOI4 medium without antibiotic for 3 days and spread out on Luria plates containing 7% sucrose. Kanamycin resistant and tetracycline sensitive clones were kept. The kanamycin marker was removed using the cre-lox system as described above. The ΔphaC mutation was confirmed by PCR and sequencing, which also revealed an additional deletion of the 5′ end of a small hypothetical gene, just upstream phaC.

### Example 2

### Analytical Methods

2.1—Poly-β-hydroxybutyrate (PHB) Analysis

PHB was quantified using the Braunegg, Sonnleitner and Lafferty method (1978) with slight modifications⁴¹⁻⁴³. Briefly, each bacterial cell culture was centrifuged at 4° C., 4000 rpm for 20 minutes. Pellets were then washed once with ice-cold water, centrifuged and lyophilised. Dry cells were resuspended using a methanolysis solution (methanol, sulfuric acid 3% and methyl benzoate 16 mM as internal standard) to obtain 5 mg of dry cells/mL. Then, 2 mL were transferred into screw cap Pyrex® glass tubes containing 2 mL of chloroform, vortexed briefly and incubated at 100° C. during 140 minutes, to allow the formation of methyl esters. During that time, tubes were vortexed occasionally. Tubes were chilled on ice and 1 mL of water was added to each reaction. Tubes were vortexed during 30 seconds and Bligh-Dyer phases were allowed to separate. The lower chloroform phases were withdrawn and PHB content was measured by gas chromatography.

### 2.2—Organic Acids Quantification

Detection and quantification of succinic acid and other carboxylic acids from the TCA cycle were performed by HPLC-UV using ICSep ICE-ION-300 column (Transgenomic), a cation-exchange polymer in the hydrogen ionic form, at a temperature of 40° C. Acidified HPLC grade water (H₂SO₄; 0.008 N) was used as the mobile phase at a flow rate of 0.4 mL/min. The analytical method used is similar to previously described methods, with slight modifications⁴⁴.

### 2.3—Microarrays

For microarrays, M. extorquens ATCC55366 and the ΔsdhA mutant were cultivated for 18-24 hours at 30° C., 250 rpm, in 50 mL of CHOI4 medium supplemented with 18.5 mM malate in the presence or absence of 0.5% (v/v) methanol. Samples were prepared as described previously⁴⁵. Briefly, immediately after cultivation, culture aliquots equivalent to 10 OD were mixed with 1/10^{th} the culture volume of cold stop solution (5% water saturated phenol, pH 7.0, 95% ethanol) and harvested at 4° C.

The cells were resuspended in 0.5 mL fresh lysosyme (3 mg/mL prepared in 10 mM Tris, 1 mM EDTA, pH 8.0) and 80 μL of 10% SDS was added. The tubes were incubated at 64° C. for 5 minutes then 88 μL 3M sodium acetate, pH 5.2 was added. Each tubes were supplemented with 800 μL prewarmed phenol:chloroform (Ambion, Burlington, Ontario), mixed by inverting the tubes and incubated at 64° C. for 6 minutes.

After cooling the tubes on ice, the samples were centrifuged at 16,000×g for 10 minutes at 4° C. to separate the phases. The aqueous phase was then mixed with the same volume of chloroform and centrifuged. The total RNA was finally precipitated with ethanol, resuspended in nuclease-free water, treated with DNase I and cleaned with RNeasy Plus Mini kit (Qiagen, Toronto, Ontario). The preparation of labeled cDNA and microarray hybridization were done exactly as described in Okubo, Y. et al (2007)⁴⁶.

Arrays were scanned using the ScanArray™ Express microarray analysis system (Perkin Elmer Life Sciences, Waltham, Mass.), and the data extracted using the ImaGene™ software (BioDiscovery Inc. Hawthorne, Calif.). Microarray data were normalized using the Lowess algorithm. Gene expression patterns were determined with GeneSpring™ visualization software version GX11 (Agilent Technologies, Santa Clara, Calif.). Gene expression levels were considered significant (p<0.05) when the fold change between strains and or conditions was more than two.

### Example 3

### The ΔsdhA Mutant

3.1—Organic Acids and PHB in the ΔsdhA Mutant

The M. extorquens wild-type strain ATCC55366 was tested and did not accumulate succinic acid when grown on methanol (data not shown). Thus, in order to achieve succinic acid build-up in cultures of M. extorquens, the sdhA gene was first knocked out as described in Example 1.1. As expected, the ΔsdhA mutant did not grow on methanol as the sole source of carbon and energy. Nevertheless, it was capable of growing in the presence of malate which rescued the TCA cycle, thereby achieving succinic acid production.

When the ΔsdhA mutant was fed only initially with 0.5% methanol, growth of the ΔsdhA mutant ceased when malic acid was completely consumed which occurred rapidly (data not shown). However, supplementing the culture with methanol throughout the course of the experiment allowed for continued growth without the addition of further malic acid. Consequently, succinic acid production by ΔsdhA mutant strain was measured while supplementing with methanol during the course of the experiment (FIG. 3). As shown in FIG. 3B, growth of the ΔsdhA mutant strain was slightly slower than the wild-type strain, but the ΔsdhA mutation allowed for succinic acid accumulation (FIG. 3A). Indeed, as shown in FIG. 3A, a concentration of 1.07 g/L (9.06 mM) of succinic acid was achieved at an optical density of 3.98 (λ=600 nm). Malic acid was consumed rapidly in the first 24 hours (from 3.11 g/L to 1.84 g/L), and was consumed more slowly afterwards. At the end of the experiment, 1.34 g/L of malic acid was still unused, giving a consumed amount of malic acid of 1.77 g/L (13.19 mM).

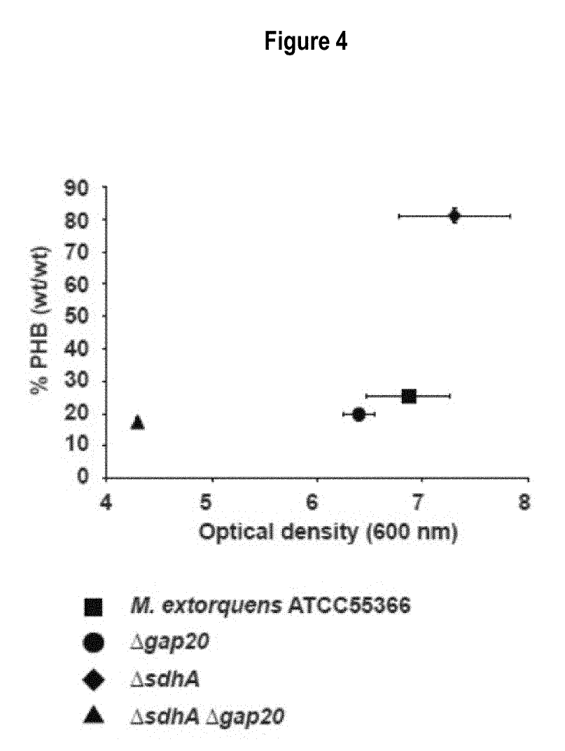

The level of competition between PHB synthesis and succinic acid production was also determined by quantifying PHB in the ΔsdhA mutant and in the wild-type strain. As shown in FIG. 4, at similar optical densities (7.3 versus 6.87), PHB concentration reached 81% (w/w) in the ΔsdhA mutant, while the wild-type ATCC55366 strain accumulated 24% (w/w).

3.1.1—ΔsdhA Mutant Fed Only Initially with 0.5% Methanol

In order to learn more about malic acid consumption, the ΔsdhA mutant was cultured for 7 days (168 h) as described above, except that 0.5% v/v methanol was added only initially without further supplementation. Cell optical density (600 nm), as well as the concentrations of succinic acid, malic acid, and methanol in the culture media were monitored over the course of the experiment.

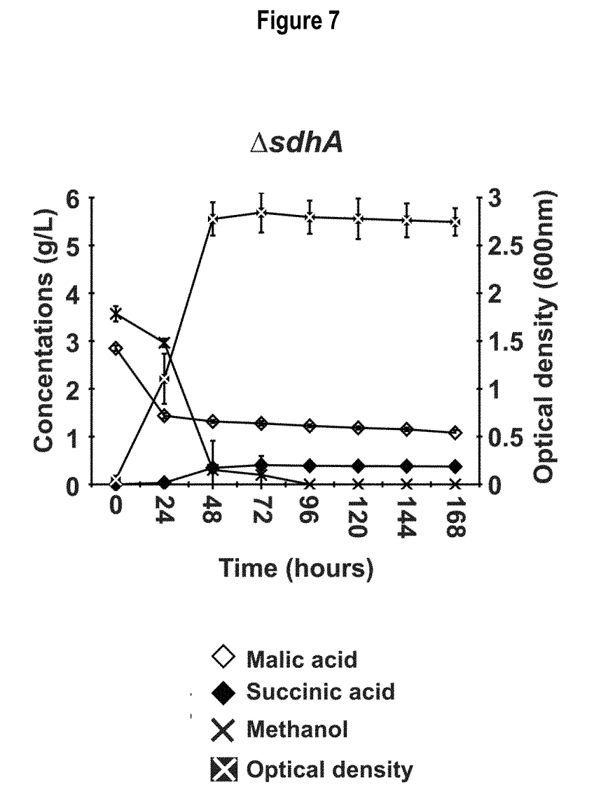

As shown in FIG. 7, growth of the ΔsdhA mutant ceased when the initially added 0.5% v/v methanol was completely consumed at about 48 hours. Succinic acid concentration reached 0.34 g/L (2.88 mM) after 48 hours and remained relatively stable until the end of the experiment.

Malic acid was rapidly consumed during the first 24 hours, and was consumed more slowly afterwards. A total of 1.77 g/L of malic acid was consumed throughout the 7-day experiment, which is a concentration greater than the concentration of succinic acid that was synthesized (FIG. 7). Because succinic acid was produced when both methanol and malic acid were available, it could not be concluded from this experiment alone that malic acid is necessary for succinic acid synthesis per se. Thus, the origin of carbon (malic acid and/or methanol) used by the ΔsdhA mutant cells to synthesize the succinic acid was unclear. Interestingly, the inflection in malic acid consumption occurred before methanol depletion and before the stationary phase (FIG. 7).

3.1.2—ΔsdhA Mutant Cultured with Periodic Methanol Supplementation
3.1.2.1—Succinic Acid Concentrations Produced by ΔsdhA Mutant

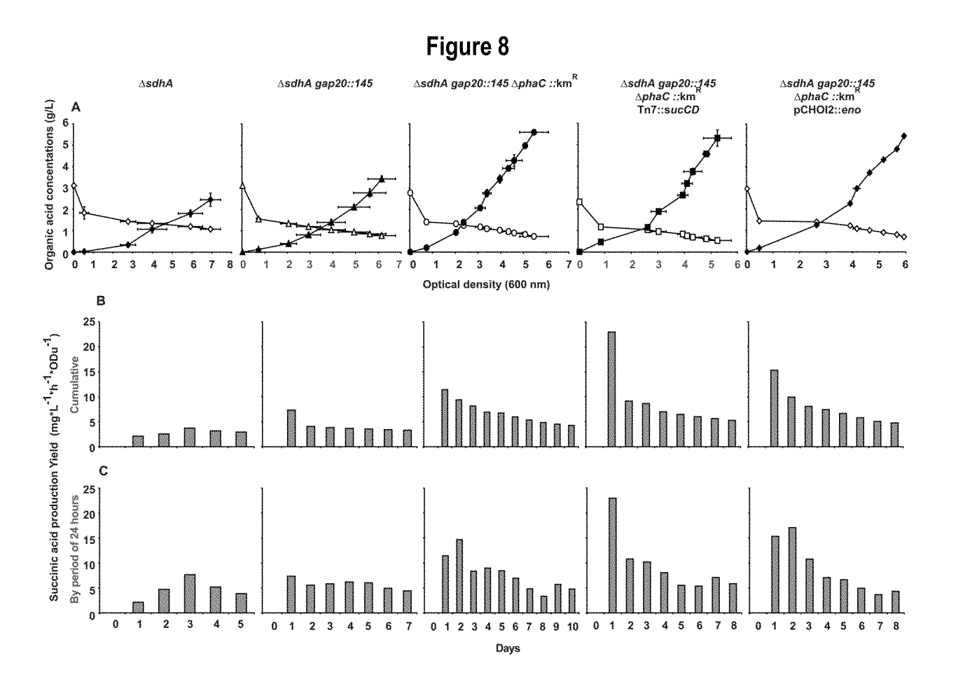

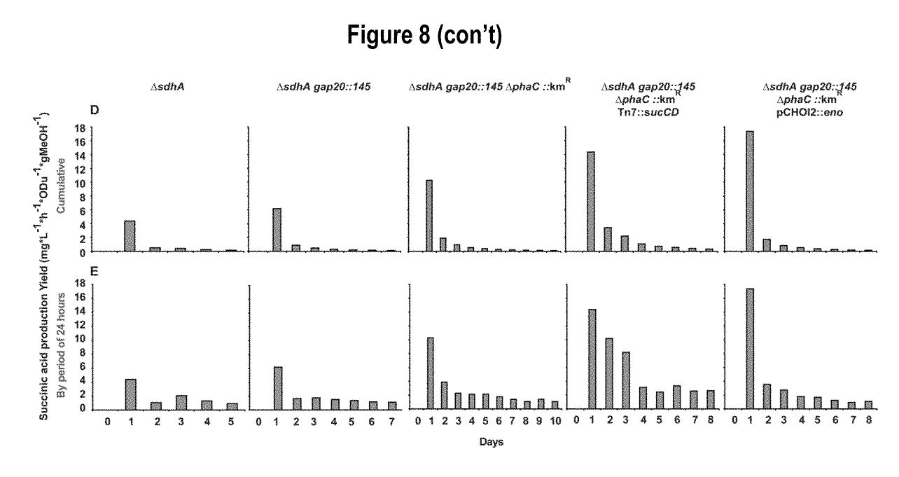

The ΔsdhA mutant was cultured for 5 days as described above in CHOI4 medium with 3 g/L (22.37 mM) of malate, while supplementing with methanol (0.5% v/v) throughout the course of the experiment. Cell optical density (600 nm), as well as the concentrations of succinic acid, malic acid, and methanol were monitored over the course of the experiment. Results are shown numerically in Table 3A and graphically in FIG. 8.

TABLE 3A
ΔsdhA mutant: Cumulative data from succinic acid production
kinetics

| | Days (cumulative) | | | | |
|---|---|---|---|---|---|
| Parameter | 1 | 2 | 3 | 4 | 5 |
| Optical density (600 nm) | 0.54 | 2.76 | 3.98 | 5.92 | 6.94 |
| Succinic acid yield (mg*L⁻¹*h⁻¹*ODu⁻¹) | 2.13 | 2.56 | 3.74 | 3.18 | 2.94 |
| Succinic acid yield (mg*L⁻¹*h⁻¹*ODu⁻¹*gMeOH⁻¹) | 4.39 | 0.52 | 0.43 | 0.25 | 0.18 |
| Succinic acid concentration (g/L) | 0.03 | 0.34 | 1.07 | 1.81 | 2.45 |
| Consumed Malic acid (g/L) | 1.28 | 1.68 | 1.77 | 1.92 | 2.04 |
| Consumed Methanol (g/L) | 2.07 | 8.88 | 12.60 | 16.57 | 16.79 |

ODu⁻¹: per optical density unit;
h⁻¹: per hour;
gMeOH⁻¹: specific yield succinic acid per gram of consumed methanol

As shown in Table 3A, the ΔsdhA mutant achieved a succinic acid concentration of 1.07 g/L (9.06 mM) at an OD of 3.98 (reached in 3 days). This point was chosen as an optical density reference to compare with subsequent experiments.

Malic acid was rapidly consumed from 3.11 g/L (23.2 mM) to 1.84 g/L (13.7 mM) in the first 24 hours, and was consumed more slowly afterwards, as observed when methanol was added only initially (Example 3.1.1). At the end of the 5-day experiment, 2.04 g/L (15.2 mM) of malic acid was consumed, which is slightly higher than the malic acid consumed without periodic methanol supplementation (1.67 g/L after 5 days; see Example 3.1.1 and FIG. 7). However, the present culture conditions reached an OD₆₀₀ of 6.94 after 5 days (Table 3.1), which is significantly higher than the OD₆₀₀ of 2.8 after 5 days observed without periodic methanol supplementation (Example 3.1.1 and FIG. 7). Furthermore, a succinic acid concentration of 2.45 g/L (20.8 mM) was achieved in this experiment, as opposed to only about 0.34 g/L without periodic methanol supplementation (FIG. 7).

Consequently, when considering the concentration of consumed malic acid, it can be deduced that at least 0.7 g/L (5.6 mM) of succinic acid must have been synthesized from methanol. Interestingly, while the periodic additions of methanol significantly improved the growth of the ΔsdhA mutant, malic acid consumption was not significantly affected in this experiment.

3.1.2.2—Cumulative Yield of Succinic Acid Produced by the ΔsdhA Mutant

At the reference optical density (˜4), the cumulative yield of succinic acid was 3.7 mg *L⁻¹*h⁻¹*ODu⁻¹ (Table 3A and FIG. 8). At the end of the experiment, an average succinic acid yield of 2.9 mg *L⁻¹*h⁻¹*ODu⁻¹ was produced.

Because of technical limitations, methanol loss due to evaporation was not quantified. Nevertheless, making the hypothesis that evaporative methanol loss was low and constant between experiments, the specific succinic acid yield per gram of consumed methanol (gMeOH) was also calculated at each time point. It was estimated at 4.39 mg of succinic acid *L⁻¹*h⁻¹*ODu⁻¹*gMeOH⁻¹ after one day (Table 3A). Yields for the following two days were estimated at 0.52 mg and 0.43 mg of succinic acid *L⁻¹*h⁻¹*ODu⁻¹*gMeOH⁻¹, respectively. Then, for the remaining days, specific yields diminished below 0.25 mg of succinic acid *L⁻¹*h⁻¹*ODu⁻¹*gMeOH⁻¹.

3.1.2.3—Yield for Each Period of 24 Hours Produced by ΔsdhA Mutant

To determine whether the ΔsdhA mutant cultures had gradually lost the capacity to produce succinic acid, as suggested by the above results, the amount of methanol consumed periodically was estimated and used to calculate the specific yield for each individual segment of 24 hours. Results are shown numerically in Table 3B and graphically in FIG. 8C.

TABLE 3B
ΔsdhA mutant: Data from succinic acid production
kinetics for each period of 24 hours

| | Days (period of 24 h) | | | | |
|---|---|---|---|---|---|
| Parameter | 1 | 2 | 3 | 4 | 5 |
| Optical density ( 600 nm) | 0.54 | 2.76 | 3.98 | 5.92 | 6.94 |
| Succinic acid yield (mg*L⁻¹*h⁻¹*ODu⁻¹) | 2.13 | 4.71 | 7.66 | 5.18 | 3.86 |
| Succinic acid yield (mg*L⁻¹*h⁻¹*ODu⁻¹*gMeOH⁻¹) | 4.39 | 1.06 | 2.06 | 1.30 | 0.93 |
| Succinic acid concentration (g/L) | 0.03 | 0.31 | 0.73 | 0.74 | 0.64 |
| Consumed Malic acid (g/L) | 1.28 | 0.40 | 0.09 | 0.15 | 0.12 |
| Consumed Methanol (g/L) | 0.49 | 4.45 | 3.72 | 3.97 | 4.16 |

The overall succinic acid yield was found to be relatively stable between periods, with only a slight decrease over time, when compared to that obtained from cumulative data (Table 3A vs Table 3B, FIG. 8B vs 8C, 8D vs 8E). These results suggest that the ΔsdhA mutant retains a significant part of its succinic acid synthesis capability over time.

3.2—ΔsdhA Mutant Transcriptomic Analysis

Transcriptomic analyses were performed on the ΔsdhA mutant strain using microarrays. Bacteria were grown in CHOI4 medium containing malate, or both malate and methanol as carbon and energy sources. Results of growth on malate confirmed the succinate dehydrogenase null phenotype of our mutant, as the sdhA and sdhB transcripts were barely detected when compared to that of the wild-type strain. In contrast, sdhC and sdhD genes were up-regulated in the mutant. Thus, in these conditions, the ΔsdhA mutation had a polar effect on the downstream genes of the operon while having a positive feedback effect on its expression. This phenomenon was not observed when the strains were grown in media supplemented with methanol, due to the weak expression of the TCA cycle genes during methylotrophic growth.

The microarray analyses also revealed that an important nutrient stress response is induced by the inactivation of the succinate dehydrogenase. Importantly, chemotaxis and flagellar genes are modulated and this is known to occur because of the fumarate concentration fluctuation⁴⁷⁻⁴⁹. These microarray results are also in accordance with stimuli known to induce PHB polymerisation^{22,27}.

When considering specifically the genes involved in methanol dissimilation and assimilation, up-regulated genes included the NADP-dependent methylene-tetrahydromethanopterin/methylene-tetrahydrofolate dehydrogenase MtdA, the methenyltetrahydrofolate cyclohydrolase Fch, and the subunit C of the formyltransferase/hydrolase complex Fhc.

Most serine cycle genes (e.g. gck, mtk) were also up-regulated, except glyA, eno and mdh, respectively encoding for serine hydroxymethyltransferase, enolase and malate dehydrogenase. The malate dehydrogenase mqo gene, however, was downregulated. HPLC tests showed that this phenomenon was not caused by oxaloacetate (OAA) accumulation in ΔsdhA mutant cultures. PHB depolymerases DepB and HbdA were also down-regulated, which is in agreement with the higher PHB content of the ΔsdhA mutant, compared to that of the wild-type strain.

No genes belonging to the EMC were found to be differentially expressed, suggesting that no feedback inhibition occurs on these genes, at the level of transcription, as a result of inactivation of sdhA and/or succinic acid accumulation. Except for sdh operon genes, no other TCA gene was differentially expressed.

### Example 4

### gap20 Mutation and PHB Synthesis

4.1—PHB Synthesis in gap20 Mutants

Even though the above results showed that the ΔsdhA mutant was able to produce succinic acid, they also demonstrated that an important proportion of the available carbon was used by the ΔsdhA mutant for the synthesis of PHB. By reducing PHB formation, carbon flux should flow through the EMC increasing succinic acid accumulation and glyoxylate synthesis, thereby reducing the need for malic acid supplementation. PHB formation/accumulation may be reduced, for example, by blocking or reducing PHB synthesis directly, or by over-expressing PHB depolymerases. For instance, inactivation or inhibition of a phasin protein or its encoding gene belongs to the first category.

As mentioned above, phasins Gap11 and Gap20 have previously been identified in M. extorquens²⁵. A mutation was thus introduced within the phasin gene gap20 (see Example 1.2). Inactivation of the gap20 gene alone in the wild-type ATCC55366 strain (using the same method as described in Example 1.2) only slightly diminished PHB accumulation, i.e. from 24% to 20% compared to ATCC55366 (w/w; FIG. 4).

Introduction of this mutation within the ΔsdhA mutant (see Example 1.2) highly reduced its PHB content, i.e. from 81% to 17%, a 4.76 fold decrease. The ΔsdhA gap20 double mutant thus produced PHB at levels comparable to the wild-type strain and the gap20 mutant (FIG. 4).

4.2—Organic Acid Synthesis in gap20 Mutants

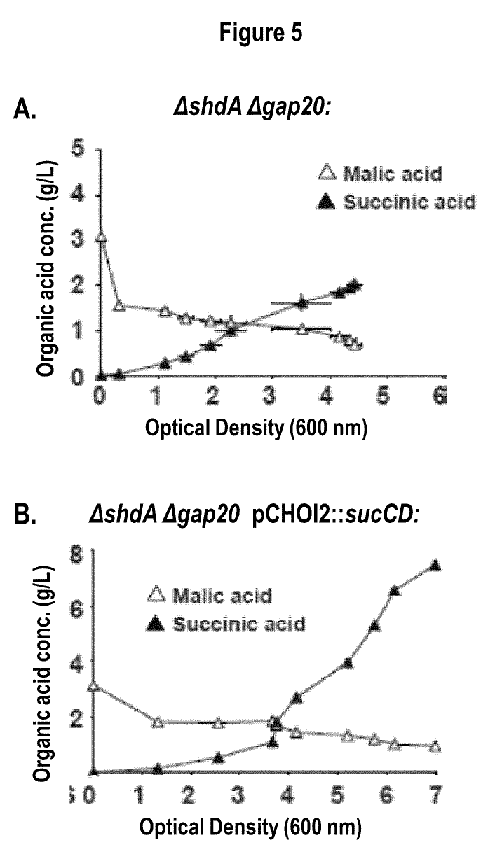

Inactivation of the gap20 gene in the ATCC55366 strain did not result in any accumulation of succinic acid. On the other hand, as shown in FIG. 5A, the ΔsdhA gap20 double mutant produced 1.4 g/L (11.86 mM) of succinic acid at an optical density of 3.93, which is about 31% greater than the amount of succinic acid produced by the ΔsdhA mutant at the same optical density (FIG. 3A). In the conditions tested, growth of the double mutant was a little slower than that of the ΔsdhA mutant. At the end of the experiment, succinic acid concentration reached 3.43 g/L (29 mM), while 0.77 g/L of malic acid was still unused. The consumed amount of malic acid was 2.35 g/L (17.53 mM; FIG. 5A). The ratio of succinic acid produced over consumed malic acid was slightly higher in the sdhA gap20 double mutant when compared to the ΔsdhA mutant.

4.3—ΔsdhA gap20 Double Mutant Cultured with Periodic Methanol Supplementation
4.3.1—Succinic Acid Concentrations Produced by ΔsdhA gap20 Double Mutant

The ΔsdhA gap20 double mutant was cultured for 5 days as described above, while supplementing with methanol (0.5% v/v) throughout the course of the experiment. Cell optical density (600 nm), as well as the concentrations of succinic acid, malic acid, and methanol were monitored over the course of the experiment. Results are shown numerically in Table 4A and graphically in FIG. 8

TABLE 4A
ΔsdhA gap 20 mutant: Cumulative data from succinic acid production kinetics

| | Days (cumulative) | | | | | | |
|---|---|---|---|---|---|---|---|
| Parameter | 1 | 2 | 3 | 4 | 5 | 6 | 7 |
| Optical density (600 nm) | 0.73 | 2.05 | 2.93 | 3.93 | 4.93 | 5.63 | 6.15 |
| Succinic acid yield (mg * L⁻¹ * h⁻¹ * ODu⁻¹) | 7.35 | 4.08 | 3.84 | 3.70 | 3.56 | 3.42 | 3.31 |
| Succinic acid yield (mg * L⁻¹ * h⁻¹ * ODu⁻¹ * gMeOH⁻¹) | 6.19 | 0.90 | 0.49 | 0.31 | 0.22 | 0.17 | 0.13 |
| Succinic acid concentration (g/L) | 0.13 | 0.40 | 0.81 | 1.40 | 2.11 | 2.78 | 3.43 |
| Consumed Malic acid (g/L) | 1.56 | 1.78 | 1.92 | 2.07 | 2.17 | 2.26 | 2.34 |
| Consumed Methanol (g/L) | 1.19 | 4.55 | 7.89 | 11.99 | 16.46 | 20.66 | 24.61 |

ODu⁻¹: per optical density unit;
h⁻¹: per hour;
gMeOH⁻¹: specific yield succinic acid per gram of consumed methanol

As shown in Table 4A, when the ΔsdhA gap20 double mutant reached an optical density of about 4, it produced 1.4 g/L (11.86 mM) of succinic acid. In contrast, when the ΔsdhA mutant reached the same optical density, it produced 1.07 g/L (9.06 mM) of succinic acid (Table 3A). However, the ΔsdhA mutant reached this optical density one day faster (3 days) and the ΔsdhA gap20 double mutant (4 days).

After 5 days, the culture of the ΔsdhA gap20 double mutant reached an OD₆₀₀ of 4.93 and a succinic acid concentration of 2.11 g/L (17.8 mM) (Table 4A). In comparison, the culture of the ΔsdhA mutant after 5 days reached a succinic acid concentration of 2.45 g/L (20.8 mM) (Table 3A). Also after 5 days, 2.17 g/L (16.27 mM) of malic acid was consumed in the culture of the ΔsdhA gap20 double mutant. Consequently, at least 0.18 g/L (1.53 mM) of succinic acid must have been synthesized from methanol by the ΔsdhA gap20 double mutant, compared to 0.7 g/L (5.6 mM) for the ΔsdhA mutant (OD₆₀₀ of 6.94).

At the end of the experiment (OD₆₀₀=6.15; 7 days), 3.43 g/L (29 mM) of succinic acid was achieved for the ΔsdhA gap20 double mutant, while the consumed amount of malic acid was 2.34 g/L (17.53 mM). Consequently, at least 1.36 g/L (11.5 mM) of succinic acid must have been synthesized from methanol.

In summary, the absolute concentrations of synthesized succinic acid that were measured at reference points (OD₆₀₀˜4 and 5 days), are lower for the ΔsdhA gap20 double mutant than for the ΔsdhA mutant. However, in the conditions tested, growth of the double mutant was a little slower than the ΔsdhA mutant and, as described below, this has had an impact on succinic acid yield measurements.

4.32—Cumulative Yield of Succinic Acid Produced by the ΔsdhA gap20 Double Mutant

At the reference optical density (OD₆₀₀ 3.93), the cumulative yield for the ΔsdhA gap20 double mutant was 3.7 mg*L⁻¹*h⁻¹*ODu⁻¹ (Table 4A and FIG. 8B), which is an amount equal to that obtained with the ΔsdhA mutant. However, as a result of growth rate differences between the ΔsdhA and ΔsdhA gap20 mutant strains, time point succinic acid yields were all shown to be higher in the double mutant. Indeed, after 5 days, 3.56 mg of succinic acid *L⁻¹*h⁻¹*ODu⁻¹ was produced by the ΔsdhA gap20 double mutant (Table 4A), compared to 2.94 mg*L⁻¹*h⁻¹*ODu⁻¹ for the sdhA mutant (Table 3A). After 7 days, 3.31 mg of succinic acid *L⁻¹*h⁻¹*ODu⁻¹ was produced.

When considering the methanol consumption, cumulative yields were also higher in the ΔsdhA gap20 double mutant than in the ΔsdhA mutant (Table 4A and FIG. 8D). As previously observed with the ΔsdhA mutant, the overall succinic acid yield was shown to rapidly decrease between 24 and 48 hours, and then more slowly decrease until the end of the experiment.

4.33—Yield for Each Period of 24 Hours Produced by the ΔsdhA gap20 Double Mutant

When considering succinic acid production as well as methanol consumption for each segment of 24 hours, the overall succinic acid yield was found to be more stable between periods, with a slow constant decrease over time, when compared to that obtained from cumulative data (Table 4B and FIG. 8D vs. 8E).

TABLE 4B
ΔsdhA gap20 mutant: Data from succinic acid production kinetics for each period of 24 hours

| | Days (period of 24 h) | | | | | | |
|---|---|---|---|---|---|---|---|
| Parameter | 1 | 2 | 3 | 4 | 5 | 6 | 7 |
| Optical density (600 nm) | 0.73 | 2.05 | 2.93 | 3.93 | 4.93 | 5.63 | 6.15 |
| Succinic acid yield (mg * L⁻¹ * h⁻¹ * ODu⁻¹) | 7.35 | 5.54 | 5.82 | 6.19 | 6.03 | 4.94 | 4.41 |
| Succinic acid yield (mg * L⁻¹ * h⁻¹ * ODu⁻¹ * gMeOH⁻¹) | 6.19 | 1.65 | 1.75 | 1.51 | 1.35 | 1.17 | 1.12 |
| Succinic acid concentration (g/L) | 0.13 | 0.27 | 0.41 | 0.58 | 0.71 | 0.67 | 0.65 |
| Consumed Malic acid (g/L) | 1.56 | 0.21 | 0.14 | 0.15 | 0.11 | 0.09 | 0.08 |
| Consumed Methanol (g/L) | 1.19 | 3.37 | 3.33 | 4.11 | 4.46 | 4.21 | 3.95 |

ODu⁻¹: per optical density unit;
h⁻¹: per hour;
gMeOH⁻¹: specific yield succinic acid per gram of consumed methanol

Except for one time point (3 days), all other specific succinic acid yields were higher in the ΔsdhA gap20 double mutant (Table 4B), compared to the ΔsdhA mutant (Table 3B). When omitting methanol consumption, yields were similar between cumulative and periodic data (Table 4A vs. Table 4B; FIG. 8B vs. 8C).

### Example 5

### The ΔsdhA gap20 ΔphaC Triple Mutant

5.1—PHB Synthesis in ΔsdhA gap20 ΔphaC Triple Mutant

A ΔphaC::Km^{R} mutation was introduced into the ΔsdhA gap20 double mutant background and the genotype of the kanamycin sensitive derivative (after Cre-Lox excision of the Km marker) was confirmed by sequencing, as described in Example 1.5. The ΔsdhA gap20 ΔphaC::Km^{R} triple mutant does not accumulate PHB, as determined by GC analyses (data not shown). The kanamycin sensitive triple mutant was used as a recipient strain for the pCHOI2 Km^{R} plasmid, as further described below. Using these PHB null mutants as cell factories, it was hypothesized that more carbon would be available for succinic acid synthesis.

5.2—ΔsdhA gap20 ΔphaC Triple Mutant with Periodic Methanol Supplementation
5.2.1—Succinic Acid Concentrations Produced by ΔsdhA gap20 ΔphaC Triple Mutant

The ΔsdhA gap20 ΔphaC triple mutant was cultured for 10 days as described above in CHOI4 medium with 3 g/L (22.37 mM) of malate, while supplementing with methanol (0.5% v/v) throughout the course of the experiment. Cell optical density (600 nm), as well as the concentrations of succinic acid, malic acid, and methanol were monitored over the course of the experiment. Results are shown numerically in Table 5A and graphically in FIG. 8.

TABLE 5A
ΔsdhA gap20 ΔphaC mutant: Cumulative data from succinic acid production kinetics

| | Days (cumulative) | | | | | | | | | |
|---|---|---|---|---|---|---|---|---|---|---|
| Parameter | 1 | 2 | 3 | 4 | 5 | 6 | 7 | 8 | 9 | 10 |
| Optical density (600 nm) | 0.74 | 2.03 | 2.37 | 3.10 | 3.39 | 3.96 | 4.34 | 4.59 | 5.07 | 5.46 |
| Succinic acid yield | 11.44 | 9.41 | 8.17 | 6.93 | 6.76 | 5.98 | 5.36 | 4.85 | 4.54 | 4.27 |
| (mg * L⁻¹ * h⁻¹ * ODu⁻¹) | | | | | | | | | | |
| Succinic acid yield | 10.28 | 1.92 | 0.95 | 0.54 | 0.40 | 0.29 | 0.22 | 0.18 | 0.14 | 0.12 |
| (mg * L⁻¹ * h⁻¹ * ODu⁻¹ * gMeOH⁻¹) | | | | | | | | | | |
| Succinic acid concentration (g/L) | 0.20 | 0.92 | 1.39 | 2.06 | 2.75 | 3.41 | 3.91 | 4.28 | 4.98 | 5.60 |
| Consumed Malic acid (g/L) | 1.41 | 1.49 | 1.57 | 1.64 | 1.72 | 1.79 | 1.85 | 1.92 | 1.99 | 2.08 |
| Consumed Methanol (g/L) | 1.11 | 4.90 | 8.57 | 12.80 | 16.75 | 20.70 | 24.16 | 27.27 | 31.35 | 35.83 |

ODu⁻¹: per optical density unit;
h⁻¹: per hour;
gMeOH⁻¹: specific yield succinic acid per gram of consumed methanol

The triple mutant produced 3.41 g/L (28.9 mM) of succinic acid at an optical density of 3.96 (6 days), which is ˜2 g/L greater than the amount of succinic acid produced by the ΔsdhA and ΔsdhA gap20 mutants, grown at the same optical density (Table 5A and FIG. 8).

Because growth of this triple mutant was even slower than that of the ΔsdhA gap20 double mutant, cultures reached an average OD₆₀₀ of only 3.39 after 5 days, whereas it achieved a succinic acid concentration of 2.75 g/L (23.3 mM), compared to 2.45 and 2.11 g/L for the ΔsdhA and ΔsdhA gap20 mutants, respectively. At this time point, 1.72 g/L (12.83 mM) of malic acid was consumed, giving a succinic acid concentration produced from methanol of at least 1.24 g/L (10.47 mM), which is more than that obtained with both ΔsdhA and ΔsdhA gap20 mutants (0.7 and 0.18 g/L respectively).

Likewise, after seven days, the average succinic acid concentration reached by the triple mutant cultures (3.91 g/L, 33.11 mM) was higher than that obtained with the ΔsdhA gap20 mutant cultures (3.43 g/L, 29 mM), though it reached a lower optical density (4.34 vs 6.15). Also, 1.85 g/L of malic acid was consumed by the triple mutant, giving a succinic acid concentration synthesized from methanol of at least 2.28 g/L (19.31 mM) versus 1.36 g/L (11.15 mM) for the ΔsdhA gap20 double mutant.

Furthermore, the triple mutant was able to produce succinic acid for a longer period of time than the ΔsdhA and ΔsdhA gap20 mutant strains, and at the end of the experiment, succinic acid concentration reached 5.60 g/L (47.47 mM) at an OD of 5.46 (10 days). Also, 2.04 g/L (15.22 mM) of malic acid was consumed. Consequently, at least 3.8 g/L (32.25 mM) of succinic acid must have been synthesized from methanol. Of note, succinic acid synthesis occurred while malic acid was only slightly consumed. Indeed, malic acid was rapidly consumed during the first 24 hours of the experiment, but was then barely consumed with an average of 0.076 g/L/24 h (Table 5A).

5.2.2—Cumulative Yield of Succinic Acid Produced by the ΔsdhA gap20 ΔphaC Mutant

At the reference OD₆₀₀ of 3.96, the triple mutant produced 5.98 mg of succinic acid *L⁻¹*h⁻¹*ODu⁻¹ of succinic acid, which is at least 2.2 mg more than the ΔsdhA or ΔsdhA gap20 mutants. At 5 days, 6.8 mg*L⁻¹*h⁻¹*ODu⁻¹ of succinic acid was produced, compared to 2.9 and 3.6 mg for the ΔsdhA and ΔsdhA gap20 mutants, respectively. After 10 days, 4.28 mg of succinic acid *L⁻¹*h⁻¹*ODu⁻¹ was achieved. Similar to results obtained with the other mutants, succinic acid yield diminished over time. However, succinic acid yields were found to be nearly two times higher in the PHB negative mutant compared to the ΔsdhA gap20 double mutant. This was also true when taking into account the methanol consumption (Table 5A and FIGS. 8B & 8D).

5.2.3—Yield for Each Period of 24 Hours Produced by the ΔsdhA gap20 ΔphaC Mutant

Similar to what was observed for the ΔsdhA gap20 double mutant, when considering 24 hour periods, the overall yield (including methanol consumption) was found to be relatively stable between periods, with a constant and slow decrease over time, when compared to that obtained from cumulative data (Table 5B; FIG. 8D vs. 8E). When omitting methanol consumption, overall, yields were similar between cumulative and periodic data (Table 5B; FIG. 8B vs. 8C). Strikingly, yields obtained with the triple mutant were higher than those of ΔsdhA and ΔsdhA gap20 mutants, regardless of methanol consumption (Table 5B; FIG. 8C vs. 8E).

TABLE 5B
ΔsdhA gap20 ΔphaC mutant: Data from succinic acid production kinetics for each period of 24 hours

| | Days (period of 24 h) | | | | | | | | | |
|---|---|---|---|---|---|---|---|---|---|---|
| Parameter | 1 | 2 | 3 | 4 | 5 | 6 | 7 | 8 | 9 | 10 |
| Optical density (600 nm) | 0.74 | 2.03 | 2.37 | 3.10 | 3.39 | 3.96 | 4.34 | 4.59 | 5.07 | 5.46 |
| Succinic acid yield (mg * L⁻¹ * h⁻¹ * ODu⁻¹) | 11.44 | 14.68 | 8.37 | 8.98 | 8.44 | 6.94 | 4.82 | 3.32 | 5.72 | 4.78 |
| Succinic acid yield | 10.28 | 3.88 | 2.28 | 2.13 | 2.14 | 1.76 | 1.39 | 1.07 | 1.40 | 1.07 |
| (mg * L⁻¹ * h⁻¹ * ODu⁻¹ * gMeOH⁻¹) | | | | | | | | | | |
| Succinic acid concentration (g/L) | 0.20 | 0.72 | 0.48 | 0.67 | 0.69 | 0.66 | 0.50 | 0.37 | 0.70 | 0.63 |
| Consumed Malic acid (g/L) | 1.36 | 0.08 | 0.08 | 0.07 | 0.08 | 0.07 | 0.07 | 0.07 | 0.07 | 0.09 |
| Consumed Methanol (g/L) | 1.11 | 3.79 | 3.67 | 4.23 | 3.95 | 3.95 | 3.46 | 3.11 | 4.08 | 4.48 |

ODu⁻¹: per optical density unit;
h⁻¹: per hour;
gMeOH⁻¹: specific yield succinic acid per gram of consumed methanol

5.3—Culture of ΔsdhA gap20 ΔphaC Mutant Strain in 3 L Shake Flask

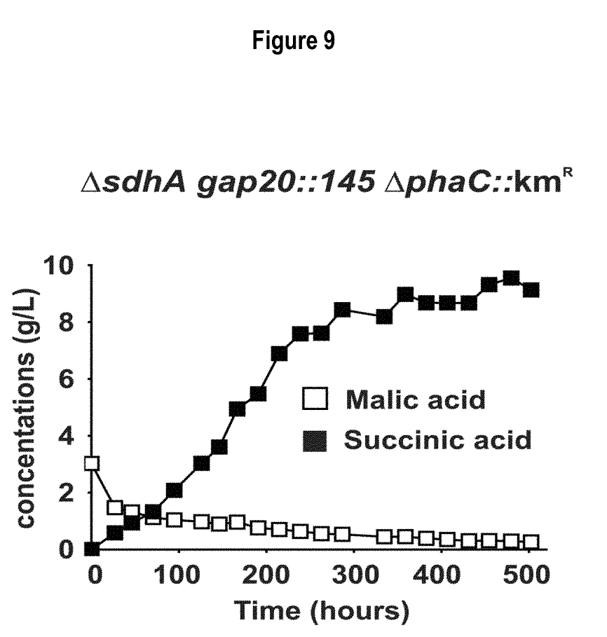

Succinic acid production in larger shake flasks was explored for the ΔsdhA gap20 ΔphaC mutant strain. The experiment in FIG. 9 is representative of two different experiments achieved using 3 L baffled Erlenmeyers, supplied in air with a pump. At the end of a 20 day experiment, succinic acid concentration reached 9.55 g/L (80.9 mM). However, at this time point, succinic acid synthesis rate was not determined because accurate OD measurements were not possible due to clumping of the culture. In this experiment, 2.73 g/L (20.34 mM) of malic acid was consumed. Thus, at least 7.15 g/L (60.6 mM) of succinic acid must have been synthesized from methanol. At 96 hours, before aggregates began to appear in the culture, succinic acid concentration reached 2.08 g/L, corresponding to 7.6 mg *L⁻¹*h⁻¹*ODu⁻¹. Consequently, this synthesis rate was faster in these conditions than those of previous experiments.

### Example 6

### Effect of sucCD Overexpression

While the inactivation of the gap20 gene reduced utilisation of malic acid by the ΔsdhA mutant, half of the amount of carbon is incorporated in succinic acid by the ΔsdhA gap20 double mutant when compared to the ΔsdhA mutant. It was found that one way of pulling even more carbon through the EMC is through the overexpression of the succinyl-CoA synthetase SucCD that oxidizes succinyl-CoA to succinate (see FIG. 1).

The sucCD genes, which overexpresses alpha and beta subunit genes (sucCD) of the succinyl-CoA synthetase, were introduced in a plasmid or within the chromosome under the P_{mxaF} promoter, using the M. extorquens ΔsdhA gap20 mutant as the recipient strain (Example 1.4). In the ΔsdhA gap20 double mutant, pCHOI2::sucCD confers a slight growth improvement as compared to the plasmid minus isogenic strain when cultured in 250 mL baffled Erlenmeyer flasks. Succinic acid production of the ΔsdhA gap20 pCHOI2::sucCD mutant strain was tested using 3 L baffled Erlenmeyer flasks. Succinic acid concentration reached 2.7 g/L (22.86 mM) at an optical density of 4.16 and 7.48 g/L (63.34 mM) at an optical density of 6.99 while the malic acid consumption reached 2.19 g/L (16.33 mM) (FIG. 5B). Even considering that all the carbon from malic acid was incorporated into succinic acid, which is unlikely, at least 47.01 mM of succinic acid must have originated solely from methanol carbon.

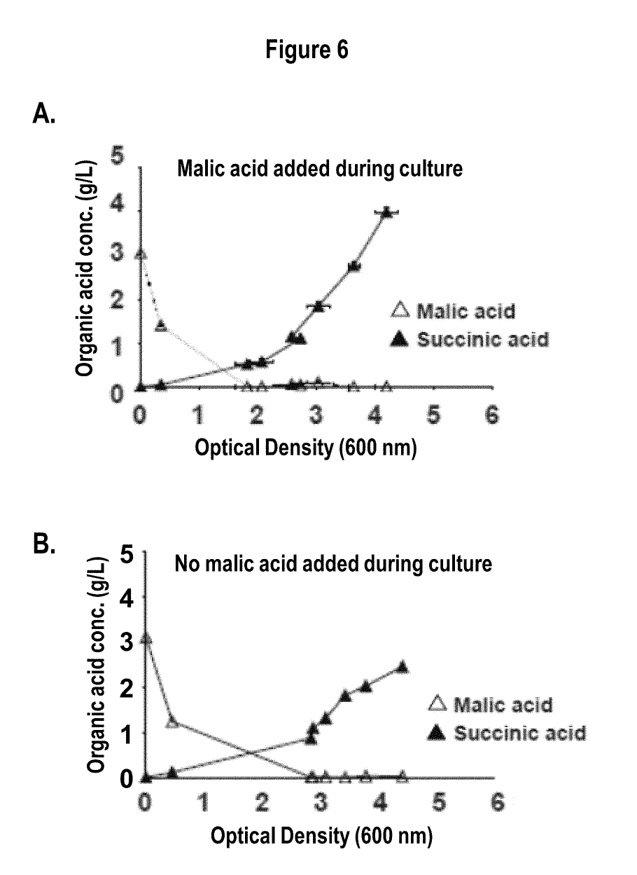

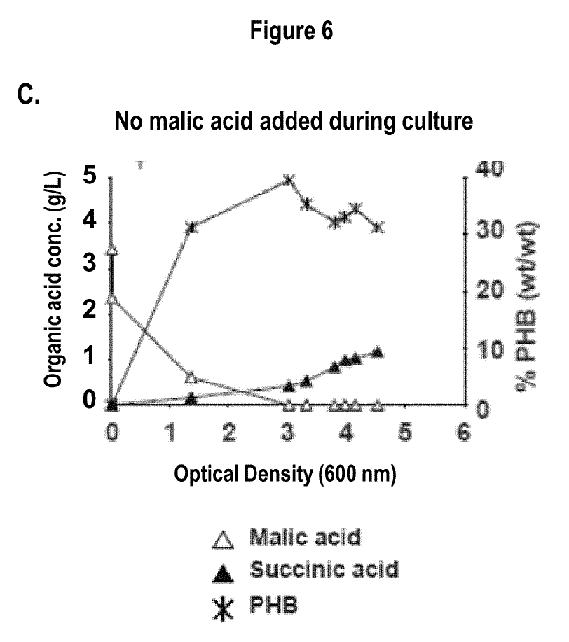

Overexpression of chromosome-integrated sucCD genes in the ΔsdhA gap20 double mutant (i.e. giving the Δsdh gap20 Tn7::sucCD strain) resulted in increased malic acid consumption by this mutant. Succinic acid concentration reached 3.99 g/L (33.79 mM) at an optical density of 4.2 when malic acid was added ad libidum (1.5 g/L every 24 h), with a total consumption of 10.56 g/L 78.73 mM) (FIG. 6A).

Succinic acid production was also further tested in ΔsdhA gap20 Tn7::sucCD cultures when supplemented with malic acid only at the start of the culture (FIG. 6B). Succinic acid concentration reached 2.45 g/L (20.75 mM) at an optical density of 4.43. Malic acid consumption was 3.1 g/L (23.01 mM). This experiment was repeated in 3 L baffled Erlenmeyer flasks. Malic acid consumption was 3.43 g/L (25.58 mM) while succinic acid concentration reached 1.18 g/L (9.99 mM), about two fold less when compared to succinic acid accumulated in the small scale experiments at similar optical densities (4.52 versus 4.43; FIG. 6C). PHB concentrations remained relatively stable at 31% (wt/wt) even though the OD readings ranged from 1.36 to 4.52 (FIG. 6C).

The Tn7::sucCD insertion is stable and the selection marker can be removed³⁹. Unexpectedly, this genetic modification abolished completely the malic acid consumption phenotype of the ΔsdhA gap20 double mutant, which consumes slowly the malic acid. Indeed, the malic acid initially added to media was completely consumed after only three days for the double mutant carrying the Tn7::sucCD fragment (FIG. 6A). The next successive additions of malic acid were also consumed rapidly and this resulted in a higher linear succinic acid production, suggesting that a part of malic acid carbon is indeed incorporated in it. Besides, succinic acid continued to be produced by the sdhA gap20 Tn7::sucCD mutant strain after complete depletion of the malic acid added from the start, without any subsequent addition of malic acid (FIG. 6B). PHB depolymerisation or reduced synthesis does not seem to be implicated in this phenomenon, as PHB cell content remained stable throughout growth in similar experiments (FIG. 6C).

### Example 7

### The ΔsdhA gap20 ΔphaC::Km^{R} Tn7::sucCD Mutant

7.1—Succinic Acid Concentrations Produced by the ΔsdhA gap20 ΔphaC::Km^{R} Tn7::sucCD Mutant

The ΔsdhA gap20 ΔphaC::Km^{R} Tn7::sucCD mutant was constructed as described in Example 6, except that the recipient strain was the ΔsdhA gap20 ΔphaC::Km^{R} triple mutant. The resulting ΔsdhA gap20 ΔphaC::Km^{R} Tn7::sucCD quadruple mutant was cultured for 8 days as described above in CHOI4 medium with 3 g/L (22.37 mM) of malate, while supplementing with methanol (0.5% v/v) throughout the course of the experiment. Cell optical density (600 nm), as well as the concentrations of succinic acid, malic acid, and methanol were monitored over the course of the experiment. Results are shown numerically in Table 6A and graphically in FIG. 8.

TABLE 6A
ΔsdhA gap20 ΔphaC::Km^{R} Tn7::sucCD mutant: Cumulative data from succinic acid production kinetics

| | Days (cumulative) | | | | | | | |
|---|---|---|---|---|---|---|---|---|
| Parameter | 1 | 2 | 3 | 4 | 5 | 6 | 7 | 8 |
| Optical density (600 nm) | 0.86 | 2.62 | 3.03 | 3.94 | 4.10 | 4.32 | 4.82 | 5.24 |
| Succinic acid yield (mg * L⁻¹ * h⁻¹ * ODu⁻¹) | 22.98 | 9.15 | 8.67 | 7.01 | 6.49 | 6.04 | 5.64 | 5.28 |
| Succinic acid yield (mg * L⁻¹ * h⁻¹ * ODu⁻¹ * gMeOH⁻¹) | 14.37 | 3.44 | 2.22 | 1.08 | 0.74 | 0.58 | 0.43 | 0.34 |
| Succinic acid concentration (g/L) | 0.47 | 1.15 | 1.89 | 2.65 | 3.20 | 3.75 | 4.57 | 5.31 |
| Consumed Malic acid (g/L) | 1.16 | 1.30 | 1.38 | 1.50 | 1.58 | 1.65 | 1.72 | 1.80 |
| Consumed Methanol (g/L) | 1.60 | 2.66 | 3.90 | 6.48 | 8.74 | 10.35 | 13.10 | 15.32 |

ODu⁻¹: per optical density unit;
h⁻¹: per hour;
gMeOH⁻¹: specific yield succinic acid per gram of consumed methanol

The ΔsdhA gap20 ΔphaC::Km^{R} Tn7::sucCD quadruple mutant, which overexpresses alpha and beta subunit genes (sucCD) of the succinyl-CoA synthetase, produced 2.65 g/L (22.44 mM) of succinic acid at an optical density of 3.94 (4 days), which is less than the amount produced by the ΔsdhA gap20 ΔphaC triple mutant alone (3.41 g/L; 28.9 mM) grown at the same optical density (Table 6A and FIG. 8A). However, growth of the sucCD overexpressing mutant was slightly faster than its parent strain.

Thus, after 5 days, the culture of the quadruple mutant reached an OD₆₀₀ of 4.1 and a succinic acid concentration 3.2 g/L (27.11 mM), compared to 2.75 g/L (23.3 mM; OD₆₀₀ 3.39) for the triple mutant. At this point, 1.72 g/L of malic acid was consumed, giving a synthesized succinic acid concentration from methanol of at least 1.69 g/L (14.29 mM), which is more than with the triple mutant at the same time point (1.24 g/L; 10.47 mM).

At the end of the experiment (8 days), succinic acid concentration reached 5.31 g/L (44.97 mM) at an OD₆₀₀ of 5.24, compared to 4.28 g/L (36.25 mM; OD600 of 4.59) for the parent triple mutant strain. The consumed amount of malic acid was 1.8 g/L (14.42 mM), giving a concentration of succinic acid that must come from methanol carbon of 3.6 g/L (30.55 mM), compared to 2.59 g/L (21.93 mM) for its parent triple mutant strain, at the same time point. Again, succinic acid synthesis occurred while malic acid was only slightly consumed (Table 6A).

7.2—Cumulative Yield of Succinic Acid Produced by the ΔsdhA gap20 ΔphaC::Km^{R} Tn7::sucCD Quadruple Mutant

At the reference OD₆₀₀ of about 4, 7.01 mg of succinic acid *L⁻¹*h⁻¹*ODu⁻¹ was produced (Table 6 and FIG. 8B). After 5 and 8 days, 6.49 and 5.28 mg of succinic acid *L⁻¹*h⁻¹*ODu⁻¹ were produced, respectively. These yields are very close to those obtained with the parent ΔsdhA gap20 ΔphaC::Km^{R} triple mutant strain, measured at the same reference points (Table 6A and FIG. 8B). Nevertheless, the sucCD overexpressing strain consumed much less methanol while it produced more succinic acid from methanol, as described above. Considering the consumed methanol, the overall specific yield of this ΔsdhA gap20 ΔphaC::Km^{R} Tn7::sucCD quadruple mutant was found to be about two fold more than that of the triple mutant. Similar to results obtained with other mutants, the overall cumulative succinic acid yield diminished over time (Table 6A and FIG. 8D).

7.3—Yield for Each Period of 24 h Produced by the ΔsdhA gap20 ΔphaC::Km^{R} Tn7::sucCD Quadruple Mutant

Strikingly, considering the methanol consumption, succinic acid yields were found to be much higher than those of the parent strain (Table 6B and FIG. 8E). Differences were especially noticeable during the first three days of growth. Furthermore, yields obtained from day 4 to day 8 with the ΔsdhA gap20 ΔphaC::Km^{R} Tn7::sucCD quadruple mutant were fairly constant, when compared to those of the triple mutant. Thus, in addition to being the best succinic acid producer obtained amongst those reported herein, the quadruple mutant also retained relatively constant productivity later during growth.

TABLE 6B
ΔsdhA gap20 ΔphaC::Km^{R} Tn7::sucCD mutant: Cumulative data from succinic acid production kinetics

| | Days (period of 24 h) | | | | | | | |
|---|---|---|---|---|---|---|---|---|
| Parameter | 1 | 2 | 3 | 4 | 5 | 6 | 7 | 8 |
| Optical density (600 nm) | 0.86 | 2.62 | 3.03 | 3.94 | 4.10 | 4.32 | 4.82 | 5.24 |
| Succinic acid yield (mg * L⁻¹ * h⁻¹ * ODu⁻¹) | 22.98 | 10.79 | 10.20 | 8.06 | 5.51 | 5.38 | 7.10 | 5.85 |
| Succinic acid yield (mg * L⁻¹ * h⁻¹ * ODu⁻¹ * gMeOH⁻¹) | 14.37 | 10.18 | 8.19 | 3.13 | 2.44 | 3.34 | 2.59 | 2.63 |
| Succinic acid concentration (g/L) | 0.47 | 0.68 | 0.74 | 0.76 | 0.54 | 0.56 | 0.82 | 0.74 |
| Consumed Malic acid (g/L) | 1.16 | 0.13 | 0.09 | 0.12 | 0.08 | 0.07 | 0.07 | 0.08 |
| Consumed Methanol (g/L) | 1.60 | 1.06 | 1.25 | 2.57 | 2.26 | 1.61 | 2.74 | 2.22 |

ODu⁻¹: per optical density unit;
h⁻¹: per hour;
gMeOH⁻¹: specific yield succinic acid per gram of consumed methanol

### Example 8

### Effect of Overexpression of the Enolase Gene eno

The construction of the plasmid containing the enolase gene eno is described in Example 1.3, and was used to overexpress the eno gene on the background of the ΔsdhA gap20 ΔphaC::Km^{R} triple mutant, giving the strain designated as ΔsdhA gap20 ΔphaC pCHOI2::eno (Km^{R}).

8.2.1—Succinic Acid Concentrations Produced by ΔsdhA gap20 ΔphaC pCHOI2::eno (Km^{R})

The ΔsdhA gap20 ΔphaC pCHOI2::eno (Km^{R}) mutant overexpressing the enolase gene eno was cultured for 8 days as described above in CHOI4 medium with 3 g/L (22.37 mM) of malate, while supplementing with methanol (0.5% v/v) throughout the course of the experiment. Cell optical density (600 nm), as well as the concentrations of succinic acid, malic acid, and methanol were monitored over the course of the experiment. Results are shown numerically in Table 7A and graphically in FIG. 8.

TABLE 7A
ΔsdhA gap20 ΔphaC pCHOl2::eno (Km^{R}) mutant: Cumulative data from succinic acid production kinetics

| | Days (cumulative) | | | | | | | |
|---|---|---|---|---|---|---|---|---|
| Parameter | 1 | 2 | 3 | 4 | 5 | 6 | 7 | 8 |
| Optical density (600 nm) | 0.48 | 2.64 | 3.90 | 4.16 | 4.64 | 5.16 | 5.67 | 5.93 |
| Succinic acid yield (mg * L⁻¹ * h⁻¹ * ODu⁻¹) | 15.33 | 9.94 | 8.08 | 7.46 | 6.68 | 5.82 | 5.07 | 4.77 |
| Succinic acid yield (mg * L⁻¹ * h⁻¹ * ODu⁻¹ * gMeOH⁻¹) | 17.37 | 1.75 | 0.84 | 0.55 | 0.38 | 0.27 | 0.20 | 0.16 |
| Succinic acid concentration (g/L) | 0.18 | 1.26 | 2.27 | 2.98 | 3.72 | 4.33 | 4.82 | 5.44 |
| Consumed Malic acid (g/L) | 1.51 | 1.56 | 1.73 | 1.87 | 1.95 | 2.05 | 2.15 | 2.26 |
| Consumed Methanol (g/L) | 0.88 | 5.69 | 9.64 | 13.59 | 17.54 | 21.49 | 25.44 | 29.39 |

ODu⁻¹: per optical density unit;
h⁻¹: per hour;
gMeOH⁻¹: specific yield succinic acid per gram of consumed methanol

The ΔsdhA gap20 ΔphaC pCHOI2::eno (Km^{R}) mutant produced 2.27 g/L (19.22 mM) of succinic acid at an optical density of 3.90 (3 days), which is less than the amount produced by the ΔsdhA gap20 ΔphaC::Km^{R} mutant (3.41 g/L; 28.9 mM; 6 days), grown at the same optical density (Table 7A and FIG. 8A). However, the growth advantage conferred by overexpression of eno was even more important than that obtained with sucCD overexpression (Example 6). This phenomenon could result from gene copy numbers, as sucCD genes were expressed into the chromosome, while the eno gene was expressed onto the medium copy plasmid pCHOI2, both constructions using the methanol dehydrogenase promoter (PmxaF). Still, during the first three days, growth rate of the eno overexpressing mutant was similar to that of the ΔsdhA mutant, and then it slowly decreased until the end of the experiment. Nevertheless, its growth rate remained always higher than that of the triple mutant, with or without sucCD overexpression. At the reference optical density, the succinic acid concentration reached was only slightly lower than that of the sucCD overexpressing strain (2.65 g/L; 22.44 mM), but it was achieved a day earlier.

After 5 days, the culture reached an OD₆₀₀ of 4.64 and a succinic acid concentration 3.72 g/L (31.5 mM). The consumed amount of malic acid was 1.95 g/L (14.54 mM), giving an amount of succinic acid synthesized from methanol of at least 2 g/L (16.94). This was more than with both the triple mutant and its isogenic derivative overexpressing sucCD (1.24 and 1.69 g/L respectively).

At the end of the experiment (8 days), succinic acid concentration reached 5.44 g/L (46.07 mM) at an OD₆₀₀ of 5.93 (8 days). The amount of consumed malic acid was 2.26 g/L (16.85 mM). Consequently, at least 3.45 g/L (29.22 mM) of succinic acid must have been synthesized from methanol. This is less than for the sucCD overexpressing strain (3.6 g/L), but more than the triple mutant alone (2.59 g/L).

8.2.2—Cumulative Yield of Succinic Acid Produced by the ΔsdhA gap20 ΔphaC pCHOI2::eno (Km^{R}) Mutant

At the reference OD (3 days), the triple mutant produced 8.08 mg of succinic acid *L⁻¹*h⁻¹*ODu⁻¹ (Table 8 and FIG. 8B). Of note, as a consequence of the growth advantage conferred by pCHOI2::eno to the ΔsdhA gap20 ΔphaC::Km^{R} triple mutant, the yield obtained at the reference optical density was found to be the highest of all experiments. Yields obtained during the five first days were found to be similar to the sucCD overexpressing mutant and its parent strain, while yields of the last three days were closer to those of the triple mutant only (Table 8 and FIG. 8B). However, the eno overexpressing mutant also showed the highest overall methanol consumption, compared to the other mutants. Consequently, when considering methanol consumption, cumulative succinic acid yields (with the exception of the 24 h time point) were all found to be slightly lower than those obtained with the triple mutant (Table 8 and FIG. 8B). Thus, succinic acid productivity is not necessarily correlated with growth rate or methanol consumption, as illustrated by results obtained with eno and sucCD overexpression.

8.2.3—Yield for Each Period of 24 Hours Produced by the ΔsdhA gap20 ΔphaC pCHOI2::eno (Km^{R}) Mutant

General observations from the triple mutant described above, were also true for its isogenic mutant overexpressing eno. Looking closer at days 2 and 3, without considering methanol consumption, periodic yields were found to be the highest of all experiments (Table 9 and FIG. 8C). After these two time points, overall, yields were found to be lower than those obtained with both the ΔsdhA gap20 ΔphaC::Km^{R} mutant and its isogenic mutant overexpressing sucCD, regardless of methanol consumption (FIGS. 8C & 8E; and Table 9). Some yields were even lower than those of the ΔsdhA gap20 double mutant, especially when considering methanol consumption. Thus, in contrast to results obtained with the sucCD overexpressing mutant, these results showed that eno overexpressing mutant lost its productivity over time, mostly at the end of experiments.

TABLE 7B
ΔsdhA gap20 ΔphaC pCHOl2::eno (Km^{R}) mutant:
Data from succinic acid production kinetics for each period of 24 hours

| | Days (period of 24 h) | | | | | | | |
|---|---|---|---|---|---|---|---|---|
| Parameter | 1 | 2 | 3 | 4 | 5 | 6 | 7 | 8 |
| Optical density (600 nm) | 0.48 | 2.64 | 3.90 | 4.16 | 4.64 | 5.16 | 5.67 | 5.93 |
| Succinic acid yield (mg * L⁻¹ * h⁻¹ * ODu⁻¹) | 15.33 | 17.07 | 10.77 | 7.07 | 6.65 | 4.93 | 3.66 | 4.31 |
| Succinic acid yield (mg * L⁻¹ * h⁻¹ * ODu⁻¹ * gMeOH⁻¹) | 17.37 | 3.55 | 2.73 | 1.79 | 1.68 | 1.25 | 0.93 | 1.09 |
| Succinic acid concentration (g/L) | 0.18 | 1.08 | 1.01 | 0.71 | 0.74 | 0.61 | 0.50 | 0.61 |
| Consumed Malic acid (g/L) | 1.51 | 0.04 | 0.18 | 0.14 | 0.08 | 0.10 | 0.10 | 0.11 |
| Consumed Methanol (g/L) | 0.88 | 4.81 | 3.95 | 3.95 | 3.95 | 3.95 | 3.95 | 3.95 |

ODu⁻¹: per optical density unit;
h⁻¹: per hour;
gMeOH⁻¹: specific yield succinic acid per gram of consumed methanol

### Summary from Examples 3-8

Table 10A below compiles the cumulative data from succinic acid production kinetics from Tables 3A, 4A, 5A, 6A, 7A and 8A, for the different mutants tested. Table 10B below compiles the data from succinic acid production kinetics for each period of 24 hours from Tables 3B, 4B, 5B, 6B, 7B and 8B, for the different mutants tested. The different mutants shown in Tables 10A and 10B are as follows:

A: ΔsdhA D: ΔsdhA gap20 ΔphaC::Km^{R} Tn7::sucCD (Tet^{R})

B: ΔsdhA gap20 E: ΔsdhA gap20 ΔphaC pCHOI2::eno (Km^{R})

C: ΔsdhA gap20 ΔphaC Km^{R}

For ease of comparison, the results after Day 5 of culture, which was the end-point of the experiment with the ΔsdhA single mutant, are shown in bold.

TABLE 10A
Complied data from cumulative succinic acid production kinetics

| Day | A | B | C | D | E | A | B | C | D | E | A | B | C | D | E |
|---|---|---|---|---|---|---|---|---|---|---|---|---|---|---|---|
| | Optical Density | | | | | Yield (mg * L⁻¹ * | | | | | Yield (mg * L⁻¹ * H⁻¹ * | | | | |
| | (λ = 600 nm) | | | | | H⁻¹ * ODu⁻¹) | | | | | ODu⁻¹ * gMeOH⁻¹) | | | | |
| 1 | 0.54 | 0.73 | 0.74 | 0.86 | 0.48 | 2.13 | 7.35 | 11.44 | 22.98 | 15.33 | 4.39 | 6.19 | 10.28 | 14.37 | 17.37 |
| 2 | 2.76 | 2.05 | 2.03 | 2.62 | 2.64 | 2.56 | 4.08 | 9.41 | 9.15 | 9.94 | 0.52 | 0.90 | 1.92 | 3.44 | 1.75 |
| 3 | 3.98 | 2.93 | 2.37 | 3.03 | 3.90 | 3.74 | 3.84 | 8.17 | 8.67 | 8.08 | 0.43 | 0.49 | 0.95 | 2.22 | 0.84 |
| 4 | 5.92 | 3.93 | 3.10 | 3.94 | 4.16 | 3.18 | 3.70 | 6.93 | 7.01 | 7.46 | 0.25 | 0.31 | 0.54 | 1.08 | 0.55 |
| 5 | 6.94 | 4.93 | 3.39 | 4.10 | 4.64 | 2.94 | 3.56 | 6.76 | 6.49 | 6.68 | 0.18 | 0.22 | 0.40 | 0.74 | 0.38 |
| 6 | | 5.63 | 3.96 | 4.32 | 5.16 | | 3.42 | 5.98 | 6.04 | 5.82 | | 0.17 | 0.29 | 0.58 | 0.27 |
| 7 | | 6.15 | 4.34 | 4.82 | 5.67 | | 3.31 | 5.36 | 5.64 | 5.07 | | 0.13 | 0.22 | 0.43 | 0.20 |
| 8 | | | 4.59 | 5.24 | 5.93 | | | 4.85 | 5.28 | 4.77 | | | 0.18 | 0.34 | 0.16 |
| 9 | | | 5.07 | | | | | 4.54 | | | | | 0.14 | | |
| 10 | | | 5.46 | | | | | 4.27 | | | | | 0.12 | | |
| | Succinic acid | | | | | Consumed Malic | | | | | Consumed | | | | |
| | concentration (g/L) | | | | | acid (g/L) | | | | | Methanol (g/L) | | | | |
| 1 | 0.03 | 0.13 | 0.20 | 0.47 | 0.18 | 1.28 | 1.56 | 1.41 | 1.16 | 1.51 | 2.07 | 1.19 | 1.11 | 1.60 | 0.88 |
| 2 | 0.34 | 0.40 | 0.92 | 1.15 | 1.26 | 1.68 | 1.78 | 1.49 | 1.30 | 1.56 | 8.88 | 4.55 | 4.90 | 2.66 | 5.69 |
| 3 | 1.07 | 0.81 | 1.39 | 1.89 | 2.27 | 1.77 | 1.92 | 1.57 | 1.38 | 1.73 | 12.60 | 7.89 | 8.57 | 3.90 | 9.64 |
| 4 | 1.81 | 1.40 | 2.06 | 2.65 | 2.98 | 1.92 | 2.07 | 1.64 | 1.50 | 1.87 | 16.57 | 11.99 | 12.80 | 6.48 | 13.59 |
| 5 | 2.45 | 2.11 | 2.75 | 3.20 | 3.72 | 2.04 | 2.17 | 1.72 | 1.58 | 1.95 | 16.79 | 16.46 | 16.75 | 8.74 | 17.54 |
| 6 | | 2.78 | 3.41 | 3.75 | 4.33 | | 2.26 | 1.79 | 1.65 | 2.05 | | 20.66 | 20.70 | 10.35 | 21.49 |
| 7 | | 3.43 | 3.91 | 4.57 | 4.82 | | 2.34 | 1.85 | 1.72 | 2.15 | | 24.61 | 24.16 | 13.10 | 25.44 |
| 8 | | | 4.28 | 5.31 | 5.44 | | | 1.92 | 1.80 | 2.26 | | | 27.27 | 15.32 | 29.39 |
| 9 | | | 4.98 | | | | | 1.99 | | | | | 31.35 | | |
| 10 | | | 5.60 | | | | | 2.08 | | | | | 35.83 | | |

TABLE 10B
Compiled from succinic acid production kinetics for each period of 24 hours

| Day | A | B | C | D | E | A | B | C | D | E | A | B | C | D | E |
|---|---|---|---|---|---|---|---|---|---|---|---|---|---|---|---|
| | Optical Density | | | | | Yield (mg * L⁻¹ * | | | | | Yield (mg * L⁻¹ * H⁻¹ * | | | | |
| | (λ = 600 nm) | | | | | H⁻¹ * ODu⁻¹) | | | | | ODu⁻¹ * gMeOH⁻¹⁾ | | | | |
| 1 | 0.54 | 0.73 | 0.74 | 0.86 | 0.48 | 2.13 | 7.35 | 11.44 | 22.98 | 15.33 | 4.39 | 6.19 | 10.28 | 14.37 | 17.37 |
| 2 | 2.76 | 2.05 | 2.03 | 2.62 | 2.64 | 4.71 | 5.54 | 14.68 | 10.79 | 17.07 | 1.06 | 1.65 | 3.88 | 10.18 | 3.55 |
| 3 | 3.98 | 2.93 | 2.37 | 3.03 | 3.90 | 7.66 | 5.82 | 8.37 | 10.20 | 10.77 | 2.06 | 1.75 | 2.28 | 8.19 | 2.73 |
| 4 | 5.92 | 3.93 | 3.10 | 3.94 | 4.16 | 5.18 | 6.19 | 8.98 | 8.06 | 7.07 | 1.30 | 1.51 | 2.13 | 3.13 | 1.79 |
| 5 | 6.94 | 4.93 | 3.39 | 4.10 | 4.64 | 3.86 | 6.03 | 8.44 | 5.51 | 6.65 | 0.93 | 1.35 | 2.14 | 2.44 | 1.68 |
| 6 | | 5.63 | 3.96 | 4.32 | 5.16 | | 4.94 | 6.94 | 5.38 | 4.93 | | 1.17 | 1.76 | 3.34 | 1.25 |
| 7 | | 6.15 | 4.34 | 4.82 | 5.67 | | 4.41 | 4.82 | 7.10 | 3.66 | | 1.12 | 1.39 | 2.59 | 0.93 |
| 8 | | | 4.59 | 5.24 | 5.93 | | | 3.32 | 5.85 | 4.31 | | | 1.07 | 2.63 | 1.09 |
| 9 | | | 5.07 | | | | | 5.72 | | | | | 1.40 | | |
| 10 | | | 5.46 | | | | | 4.78 | | | | | 1.07 | | |
| | Succinic acid | | | | | Consumed Malic | | | | | Consumed | | | | |
| | concentration (g/L) | | | | | acid (g/L) | | | | | Methanol (g/L) | | | | |
| 1 | 0.03 | 0.13 | 0.20 | 0.47 | 0.18 | 1.28 | 1.56 | 1.36 | 1.16 | 1.51 | 0.49 | 1.19 | 1.11 | 1.60 | 0.88 |
| 2 | 0.31 | 0.27 | 0.72 | 0.68 | 1.08 | 0.40 | 0.21 | 0.08 | 0.13 | 0.04 | 4.45 | 3.37 | 3.79 | 1.06 | 4.81 |
| 3 | 0.73 | 0.41 | 0.48 | 0.74 | 1.01 | 0.09 | 0.14 | 0.08 | 0.09 | 0.18 | 3.72 | 3.33 | 3.67 | 1.25 | 3.95 |
| 4 | 0.74 | 0.58 | 0.67 | 0.76 | 0.71 | 0.15 | 0.15 | 0.07 | 0.12 | 0.14 | 3.97 | 4.11 | 4.23 | 2.57 | 3.95 |
| 5 | 0.64 | 0.71 | 0.69 | 0.54 | 0.74 | 0.12 | 0.11 | 0.08 | 0.08 | 0.08 | 4.16 | 4.46 | 3.95 | 2.26 | 3.95 |
| 6 | | 0.67 | 0.66 | 0.56 | 0.61 | | 0.09 | 0.07 | 0.07 | 0.10 | | 4.21 | 3.95 | 1.61 | 3.95 |
| 7 | | 0.65 | 0.50 | 0.82 | 0.50 | | 0.08 | 0.07 | 0.07 | 0.10 | | 3.95 | 3.46 | 2.74 | 3.95 |
| 8 | | | 0.37 | 0.74 | 0.61 | | | 0.07 | 0.08 | 0.11 | | | 3.11 | 2.22 | 3.95 |
| 9 | | | 0.70 | | | | | 0.07 | | | | | 4.08 | | |
| 10 | | | 0.63 | | | | | 0.09 | | | | | 4.48 | | |

### Example 9

### Genetic Switches

To eliminate the need of malate addition to produce succinic acid using M. extorquens growth on methanol, the sdhA mutant is complemented by incorporating an sdh operon under the control of a genetic switch, into the background of the ΔsdhA mutant.

Cumate-dependent genetic switches were first described and successfully used in M. extorquens^{50,51}. Since cumate is an inexpensive molecule, it is reasonable to consider its use in bioreactors.

The switches are based on the Pseudomonas putida repressor CymR or on the chimeric transactivator cTA. cTA consists in a fusion between CymR and the activation domain of the VP16 protein (herpes simplex). These two transcriptional regulators bind to specific operator sequences. The presence of cumate prevents the binding of CymR and cTA to the operator sequence, resulting in activation or repression, respectively.

Similarly, CymR-dependent switches are also used. Cumate is then used at low concentrations to permit temporary complementation for biomass production. Sdh proteins produced from such switches are expected to be exhausted later on during growth and succinic acid would then accumulate.

As the genetic switches can be activated at any moment during growth, the engineered strains may yield higher biomass resulting in higher succinic acid production. Moreover, it is important to underline that CymR and cTA-dependent switches could theoretically be modulated by the addition of cumate generating opposite regulation effects. Likewise, it would be possible to operate these two kinds of switches together in a single mutant strain, controlling simultaneously the expression of the sdh operon and PHB depolymerisation.

Results showed that the ΔsdhA mutant carrying a genetic switch capable of controlling expression of the sdh operon can grow without malic acid supplementation in the presence of cumate. Furthermore, its growth was reduced as more cumate was added.

### Example 10

### Heterologous Genes Expression

Simultaneous overexpression of pyc and ppc genes was shown to improve aerobic succinate production in some bacteria^{54,55}. These genes encode pyruvate and phosphoenolpyruvate (PEP) carboxylases, respectively, responsible for the conversion of pyruvate and PEP into OAA. The increase of the OAA pool within M. extorquens cells is expected to provide more carbon input into the EMC, especially if the mdh gene is functionally overexpressed.

As the pyc gene is missing from the M. extorquens genome, its heterologous expression is therefore needed. For example, the pyc gene from Rhodopseudomonas palustris BisA53 is used. Like M. extorquens, R. palustris is an environmental non-pathogenic bacteria belonging to the rhizobiale group of alphaproteobacteria⁵⁶.

Likewise, heterologous overexpression of an isocitrate lyase (Icl), which catalyzes the formation of glyoxylate and succinate from isocitrate may be performed^{57,58}. Icl is a key enzyme of the glyoxylate regeneration pathway and is absent from the M. extorquens genome, which uses the EMC. For example, Icl may be used to increase the oxidative flux from citrate within the TCA, which may occur as succinic acid accumulates in a genetic switch complemented ΔsdhA mutant. It could also allow inactivation of the EMC, which theoretically, would result in larger amount of carbon available for succinic acid production.

### Example 11

### Converting PHB Accumulated Biomass to Succinic Acid

PhaC mutants were shown to have a growth defect when grown on methanol. However, unidentified suppressor mutations of this specific phenotype also occur at high frequency on methanol³¹. Therefore, a shdA phaC double mutant that does not produce PHB, but grows normally on methanol may be obtained.

Alternatively, in order to make the carbon accumulated in the PHB available for succinic acid synthesis in the ΔsdhA mutant, PHB depolymerases and recycling enzymes are cloned, alone or in combination, under an inducible promoter. PHB depolymerisation may then be induced at any time, for example when a culture reaches mid-stationary phase of growth. AtoCD activity results in the production of succinic acid as by-product during PHB depolymerisation. It may thus be possible to perform a two phase bioprocess in which PHB accumulates in a first phase and succinic acid is produced subsequently.

### Example 12

### Small RNA Knockdowns

Since the early 2000's, classes of RNA regulators have been discovered and shown to play a key role in the control of genes through various mechanisms, whether during transcription, translation or even post-translation. An important group of these regulators is composed of so-called “small RNAs” (sRNA). These genes are transcribed as short (˜100 bases) RNAs not encoding for any protein. Instead, these sRNAs can bind to target mRNAs through base complementarity, typically in the region of the ribosome binding site. Binding of the sRNA to its target prevents accessibility of the ribosome, therefore repressing translation and, consequently, expression. Close to a hundred sRNAs have been identified in Escherichia coli as well as in other species, especially proteobacteria^{59,60}.

Most sRNAs bind the protein Hfq which serves as a facilitator for the interaction between the sRNA and the mRNA to be inhibited, thus allowing more efficient binding and repression. More specifically, Hfq binds a region of the sRNA, while the other part of the sRNA can bind to the target mRNA. It is thus possible to design modified sRNAs capable of repressing any selected target⁵². While about a hundred are known in E. coli, a few sRNAs have been found so far in M. extorquens PA1, but there are likely as many as in E. coli⁶¹. Indeed, this specie harbors the hfq gene, indicator of sRNA regulatory pathways⁵⁹. Therefore, a sRNA such as MicC should function in M. extorquens as it does in E. coli, provided that it has the appropriate sequence to form base pairs with its target mRNA. For instance, “sRNA constructs” consist in a promoter, a variable region complementary to the target gene, a MicC sequence and a terminator, for a total of less than 500 bases.

Results based on GFP expression indicate that a version of the PmxaF promoter consisting of 242 bases upstream of the transcription start site is sufficient to produce a sRNA with almost no extra sequence, for instance, only a single “G” in 5′ of the sRNA “target-complementarity-region”. The sRNA system in M. extorquens can then be assayed against GFP as a reporter gene. To assess GFP repression, three anti-GFP sRNAs are constructed, these are complementary to positions −19 (relative to the start codon) up to the start codon, positions −11 to +10 and from the start codon up to +20. These sRNAs expressed by the truncated PmxaF target a genome insertion of GFP in M. extorquens, also under the control of PmxaF. Based on the results obtained, a sRNA construct complementary to sdhA is then designed. Succinic acid production using M. extorquens modified with this sRNA is measured as previously described, with and without malic acid supplementation.

Other sRNA may be designed to target other genes which encode proteins involved in the metabolism of succinic acid or which may divert intermediary metabolites from the main path linking methanol to succinate. For instance, genes involved in the citric acid cycle (e.g. sdhBCD and fumC) as well as other pathways, such as the pentose phosphate pathway (e.g. pgm, pgk, gap . . . ), the PHB pathway, or the formate oxidation pathway. Combinations of sRNAs within the same vector may also be used to increase succinic acid production. For instance, another sRNA may be combined with the sdhA sRNA or may be expressed in an sdhA mutant herein described.

Alternatively, a modified CRISPR system could also be used in a very similar way. The CRISPR RNAs are bacteria's natural defense mechanisms against bacteriophages, but can be adapted to target a gene and its functionality is irrelevant to the species in which they are used, provided that a modified Cas9 protein is co-expressed⁵³.

### Example 13

### Expression of a Heterologous Glyxoxylate Shunt and Ethyl-Malonyl-CoA Pathway Inactivation in a Methylobacterium Extorquens Mutant that Produces Succinic Acid from Methanol

### 13.1—Rationale

In cells, acetyl-CoA is a major anaplerotic metabolite and assimilation pathways have evolved to maximize its carbon incorporation into the central metabolism. In many organisms, one strategy involves the utilization of both the TCA and the Glyoxylate cycles. Indeed, acetyl-CoA can be condensed with oxaloacetate to produce citrate, thereby beginning the oxidative TCA cycle. Then, instead of being further decarboxylated, the isocitrate produced by the TCA cycle can be taken up by the Glyoxylate cycle to form succinate and glyoxylate. This last step is achieved by the isocitrate lyase enzyme (icl). Next, glyoxylate can be used together with acetyl-CoA to produce malate, making the missing carbon to enter the central metabolism.

Methylotrophic microorganisms, such as Methylobacterium extorquens, lack the Icl enzyme (the glyoxylate shunt) and use the Ethyl-Malonyl-CoA (EMC) pathway to produce, among other molecules, glyoxylate. This glyoxylate is intended to be used by the Serine Cycle for assimilation of methanol, and not for the synthesis of malate, while methanol can be the sole source of carbon and energy. Acetyl-CoA produced by the serine cycle is used as the primary substrate for the EMC pathway. This pathway involves successive thio-ester-CoA molecule modifications and flows into the TCA cycle by forming succinyl-CoA. In fact, during methylotrophic growth, the TCA cycle works only partially and enzymatic reactions toward malate synthesis complete the EMC pathway. The EMC pathway shares its two first steps with the PHB cycle, i.e. the sequential synthesis of aceto-acetyl-CoA and hydroxybutyryl-CoA (OHB-CoA) from acetyl-CoA, by PhaA (a beta-ketothiolase) and PhaB (an NADPH-linked acetoacetyl-CoA reductase), respectively. The final step of PHB synthesis is performed by the PHB synthase PhaC.

Accordingly, since EMC requires a lot of carbon and the eventual recombinant glyoxylate shunt would produce succinic acid as well as glyoxylate, overexpression of a heterologous shunt within a metabolically modified M. extorquens that produces succinic acid is herein described. Once the glyoxylate shunt is operational, the inefficient and unnecessary EMC may then be inactivated.

13.2—Autosomal Expression of a Heterologous Glyxoxylate Shunt in a M. Extorquens Mutant that Produces Succinic Acid from Methanol

This example describes the creation of a classic glyoxylate shunt within an isocitrate lyase (icl) negative M. extorquens triple mutant (ΔsdhA gap20 ΔphaC) and the assessment of its functionality. This must be performed prior to EMC pathway inactivation (Example 13.3), as it will replace an essential glyoxylate producing pathway by another. Furthermore, since the first steps of the Citrate cycle are poorly expressed in M. extorquens during growth on methanol, genes leading to isocitrate synthesis (gltA and acnA) will be overexpressed together with the isocitrate lyase shunt (icl gene). These genes encode a citrate synthase and an aconitase, respectively.
A—For the isocitrate lyase shunt, two approaches are assayed: (1) The icl gene from the environmental bacterium Rhodopseudomonas palustris BisA53 is PCR amplified and cloned within the medium copy vector pCHOI2. This plasmid is characterized by a multiple cloning site located just downstream of the strong M. extorquens methanol dehydrogenase promoter (PmxaF). The R. palustris genome is chosen as a template since the codon usage in this microorganism is similar to that of M. extorquens. (2) A custom, completely synthetic icl gene (also driven by PmxaF) is designed based on the amino acid sequence of isocitrate lyase in Escherichia coli K12 and on codon usage in M. extorquens. For gltA and acnA genes, M. extorquens ATCC55366 chromosome may be used as a template for PCR amplifications. Then, gltA and acnA are cloned into pCHOI2 vector.
B—Each plasmid insertion, together with PmxaF, is sequenced and transformed into the triple mutant. Expression of icl, gltA, acnA and rpoD (housekeeping) genes are evaluated by semi-quantitative RT-PCR, as described in the table below. This experimental design permits observation of the influence of the overexpressed gene on the expression of selected others. Expression of icl R.p. is compared to that of icl synth. Citrate synthase, aconitase and isocitrate lyase activity is also measured. The triple mutant carrying the empty pCHOI2 vector is used as a control. Experiments are performed using 250 mL baffled shake flasks, in biological triplicates. The sampling is performed during the exponential phase of growth.

RT-PCR design

| ΔsdhA gap20::145 ΔphaC derivatives | Targeted genes |
|---|---|
| +pCHOI2 (negative or basal | rpoD icl R.p. icl synth. |
| expression control) | gltA acnA |
| +pCHOI2::icl R.p. | rpoD icl R.p. gltA acnA |
| +pCHOI2::icl synthetic | rpoD icl synth. gltA acnA |
| +pCHOI2::gltA | rpoD gltA acnA |
| +pCHOI2::acnA | rpoD gltA acnA |

13.3—In Chromosome Expression of a Heterologous Glyxoxylate Shunt within an M. Extorquens Mutant that Produces Succinic Acid from Methanol

This example describes integration of the best (stronger) isocitrate lyase overexpressing system, as determined in Example 13.2, together with gltA and acnA systems, into the chromosome of the triple mutant (ΔsdhA gap20 ΔphaC) and the subsequent characterization.
A—Targeted DNA fragments are PCR amplified from pCHOI2 derived vectors obtained in Example 13.2 and integration is performed using suicide vector or Tn7-based system. Selection markers are removed using the Cre-LoxP or the flipase (Flp) system. All integrations are verified by sequencing.
B—Expression of icl, gltA, acnA and rpoD (housekeeping) are evaluated by semi-quantitative RT-PCR, as described in the table below. Citrate synthase, aconitase and isocitrate lyase activity are measured. The triple mutant is used as a control. Experiments are performed using 250 mL baffled shake flasks, in biological triplicates. The sampling is performed during the exponential phase of growth.

RT-PCR design

| Mutant strains | Targeted genes |
|---|---|
| ΔsdhA gap20::145 ΔphaC mutant (basal expression control) | rpoD gltA acnA |
| ΔsdhA gap20::145 ΔphaC // ::pmxaF-icl, pmxaF-gltA and | rpoD icl R.p. or synth. gltA acnA |
| pmxaF-acnA | |

C—Growth of the triple mutant, overexpressing or not the glyoxylate shunt, is performed using a 1.5 L DASGIP parallel bioreactor systems equipped with a methanol control system. Two reactors are used for each mutant, and runs are to last 72 h. Expression of icl, gltA and acnA are evaluated by semi-quantitative RT-PCR. Citrate synthase, aconitase and isocitrate lyase activity are measured. Succinic and malic acids are quantified by HPLC. Sampling is done every 24 h.

### 13.4—Inactivation of the EMC Pathway in the M. Extorquens Mutants Expressing the Heterologous Glyoxylate Shunt

This example describes the interruption of the EMC pathway within the mutant obtained in Example 13.3, and the characterization thereof.
A—Inactivation of the phaA gene and consequently of the entire EMC pathway is performed using a suicide vector. Selection markers are removed using the Cre-LoxP system. The phaA mutation is verified by sequencing.
B—The EMC-negative mutant is grown using a 1.5 L DASGIP parallel bioreactor system equipped with a methanol control system (3 reactors). For comparative purposes, a fourth reactor is used to grow the EMC positive isogenic mutant. Runs are to last 72 h. Expression of icl, gltA, acnA and rpoD are evaluated by semi-quantitative RT-PCR, as described in the table below. Of note, expression of two genes from the EMC pathway are also be quantified by RT-PCR. Citrate synthase, aconitase and isocitrate lyase activity are measured. Succinic and malic acids are quantified by HPLC. Sampling is done every 24 h.

RT-PCR design

| Mutant strains | Targeted genes |
|---|---|
| ΔsdhA gap20::145 ΔphaC // ::pmxaF-icl, pmxaF-gltA | rpoD, icl R.p. or synth. gltA, acnA, EMC gene 1, EMC |
| and pmxaF-acnA (basal expression control) | gene 2 |
| ΔsdhA gap20::145 ΔphaC ΔphaA // ::pmxaF-icl, pmxaF- | rpoD icl R.p. or synth. gltA, acnA, EMC gene 1, EMC |
| gltA and pmxaF-acnA | gene 2 |

Alternatively, since PhaA works upstream of both Gap20 and PhaC in the EMC pathway, inactivating PhaA may be sufficient to inactive both the PHB and EMC pathways, without having to also inactivate Gap20 and PhaC. For example, a ΔsdhA ΔphaA mutant could be created that overexpresses isocitrate lysase, and thus produce a mutant having disrupted PHB and EMC pathways, and an operational glyoxylate shunt.

It is understood that the examples and embodiments described herein are for illustrative purposes only and that various modifications or changes in light thereof will be suggested to persons skilled in the art and are to be included within the spirit and purview of this application and scope of the appended claims. Any publication, document, patent, patent application or publication referred to herein should be construed as incorporated by reference each in their entirety and for all purposes.

### REFERENCES

1. http://digital.library.wisc.edu/1711.dl/HistSciTech.Cyclopaedia01 (Vol. 1, page 75)
2. Werpy, P. and G. Petersen, Top value added chemicals from biomass. Volume 1: Results of screening potential candidates from sugars and synthesis gas. US Department of Energy, 2004.
3. Cornils, B. and P. Lappe, Dicarboxylic Acids, Aliphatic. Ullmann's Encyclopedia of Industrial Chemistry., 2000.
4. Ochsner, A. M., et al., Methylobacterium extorquens: methylotrophy and biotechnological applications. Appl Microbiol Biotechnol, 2014.
5. Trotsenko, Y. A., et al, Microbiology, 2001. 70: p. 623-632.
6. Anthony, C., The biochemistry of methylotrophs. Academic Press, 1982.
7. Hanson, R. S. and T. E. Hanson, Microbiol Rev, 1996. 60(2): p. 439-71.
8. Hwang, I. Y., et al., Batch conversion of methane to methanol using Methylosinus trichosporium OB3b as biocatalyst. J Microbiol Biotechnol, 2015.
9. Anthony, C., Science Progress, 2011. 94(2): p. 109-132.
10. Chistoserdova, L., et al., J Bacteriol, 2003. 185(10): p. 2980-7.
11. Crowther, G. J., et al, J Bacteriol, 2008. 190(14): p. 5057-62.
12. Lidstrom, M. E., FEMS Microbiol Rev, 1990. 7(3-4): p. 431-6.
13. Peyraud, R., et al., Proc Natl Acad Sci U S A, 2009. 106(12): p. 4846-51.
14. Peyraud, R., et al., PLoS One, 2012. 7(11): p. e48271.
15. Peyraud, R., et al., BMC Syst Biol, 2011. 5: p. 189.
16. Skovran, E., et al., PLoS One, 2010. 5(11): p. e14091.
17. Smejkalova, H., et al, PLoS One, 2010. 5(10): p. e13001
18. Vuilleumier, S., et al., PLoS One, 2009. 4(5): p. e5584.
19. Vorholt, J. A., Arch Microbiol, 2002. 178(4): p. 239-49.
20. Chistoserdova, L., Environ Microbiol, 2011. 13(10): p. 2603-22.
21. Schneider, K., et al., J Biol Chem, 2012. 287(1): p. 757-66.
22. Potter, M. and A. Steinbuchel, Biomacromolecules, 2005. 6(2): p. 552-60.
23. Bourque, D., et al, Appl Environ Biotechnol, 1995. 44: p. 367-376.
24. Föllner, C. G., et al, Can J Microbiol, 1995. 41(13): p. 124-130.
25. Korotkova, N., et al, J Bacteriol, 2002. 184(22): p. 6174-81.
26. Grage, K., et al., Biomacromolecules, 2009. 10(4): p. 660-9.
27. Jendrossek, D. and D. Pfeiffer, Environ Microbiol, 2014. 16(8): p. 2357-73.
28. Füller, R. C., et al., FEMS Microbiol Rev, 1992. 103: p. 279-288.
29. Liebergesell, et al, FEMS Microbiol Lett, 1992. 78(2-3): p. 227-32.
30. Pieper-Furst, U., et al., J Bacteriol, 1994. 176(14): p. 4328-37.
31. Korotkova, N. and M. E. Lidstrom, J Bacteriol, 2001. 183(3): p. 1038-46.
32. Follner, C. G., et al., J Basic Microbiol, 1997. 37(1): p. 11-21.
33. Forster, A. H. and J. Gescher, Front Bioeng Biotechnol, 2014. 2: p. 16.
34. Marx, C. J. and M. E. Lidstrom, Biotechniques, 2002. 33(5): p. 1062-7.
35. Roland, K., et al, Avian Dis, 1999. 43(3): p. 429-41.
36. Marx, C. J. and M. E. Lidstrom, Microbiology, 2001. 147(Pt 8): p. 2065-75.
37. Choi, Y. J., et al., Appl Environ Microbiol, 2010. 76(15): p. 5058-66.
38. Brosius, J., DNA, 1989. 8(10): p. 759-77.
39. Choi, K. H., et al., Nat Methods, 2005. 2(6): p. 443-8.
40. Choi, Y. J., et al., Appl Environ Microbiol, 2006. 72(1): p. 753-9.
41. Braunegg, G., et al, European J. Appl. Microbiol. Biotechnol., 1978. 6: p. 29-37.
42. Kato, M., et al., Appl Environ Biotechnol, 1996. 45: p. 363-370.
43. Orita, I., et al., Appl Microbiol Biotechnol, 2014, 98(8): p. 3715-25.
44. Zheng, Z., et al., Microb Cell Fact, 2012. 11: p. 37.
45. Khodursky, A. B., et al., Methods Mol Biol, 2003. 224: p. 61-78.
46. Okubo, Y., et al., OMICS, 2007. 11(4): p. 325-40.
47. Eisenbach, M. and S. R. Caplan, Curr Biol, 1998. 8(13): p. R444-6.
48. Montrone, M., et al., J Bacteriol, 1998. 180(13): p. 3375-80.
49. Prasad, K., et al, J Mol Biol, 1998. 280(5): p. 821-8.
50. Choi, Y. J., et al., Appl Environ Microbiol, 2006. 72(12): p. 7723-9.
51. Mullick, A., et al., BMC Biotechnol, 2006. 6: p. 43.
52. Na, D., et al., Nat Biotechnol, 2013. 31(2): p. 170-4.
53. Qi, L. S., et al., Cell, 2013. 152(5): p. 1173-83.
54. Litsanov, B., et al., Microb Biotechnol, 2012. 5(1): p. 116-28.
55. Thakker, C., et al., J Biotechnol, 2011. 155(2): p. 236-43.
56. Oda, Y., et al., Proc Natl Acad Sci U S A, 2008. 105(47): p. 18543-8.
57. Lin, H., et al, Metab Eng, 2005. 7(2): p. 116-27.
58. Zhu, N., et al., Biotechnol Lett, 36, 3, pp 553-560 (online pub. October 2013).
59. Lalaouna, D., et al., Biochim Biophys Acta, 2013. 1829(6-7): p. 742-7.
60. Waters, L. S. and G. Storz, Cell, 2009. 136(4): p. 615-28.
61. Li, L., et al., Nucleic Acids Res, 2013. 41(database issue): p. D233-8.

1.-50. (canceled)

51. A method for producing a succinate-containing fermentation broth, said method comprising growing a genetically engineered serine cycle methylotroph or methanotroph in the presence of one or more C1-compound(s) under conditions enabling the production of succinate, wherein the methylotroph or methanotroph is genetically modified to disrupt a gene encoding a tricarboxylic acid (TCA) cycle succinate dehydrogenase or a subunit thereof, to overexpress a TCA cycle succinyl-CoA synthetase, and to disrupt poly-β-hydroxybutyric acid (PHB) biosynthesis in the methylotroph or methanotroph.

52. The method of claim 51, wherein the disruption of PHB biosynthesis comprises disruption of one or more enzymes in the Ethyl-Malonyl-CoA (EMC) pathway.

53. A genetically engineered C1-utilizing microorganism that produces succinate from a C1-carbon source, the microorganism being genetically modified to disrupt a gene encoding a tricarboxylic acid (TCA) cycle succinate dehydrogenase (Sdh) or a subunit thereof.

54. The microorganism of claim 53, wherein said microorganism is:
(i) a serine cycle methylotroph from the genera Burkholderia, Fulvimarina, Granulibacter, Hyphomicrobium, Methylibium, Methylobacterium, Ruegeria, or Methylobacterium;
(ii) a serine cycle methylotroph not mentioned in (i);
(iii) a serine cycle methanotroph from the genera Methanomonas, Methylocystis, Methylocapsa, Methylocella, Methylococcus, Methylosinus, or Methylosinus; or
(iv) a serine cycle methanotroph not mentioned in (iii).

55. The microorganism of claim 53, which is genetically modified:
(a) by the knockout, knockdown or deletion of an sdh gene;
(b) to inhibit, reduce or eliminate the activity of a protein involved in polyhydroxyalkanoate or poly-β-hydroxybutyric acid (PHB) biosynthesis and/or granule homeostasis;
(c) to overexpress a TCA cycle succinyl-CoA synthetase;
(d) to express or overexpress a isocitrate lyase and/or a protein involved in isocitrate synthesis;
(e) to inhibit, reduce, or eliminate the activity of a protein involved in the Ethyl-Malonyl-CoA (EMC) pathway; or
(f) any combination of (a) to (e).

56. The microorganism of claim 55, wherein:
(a) the sdh gene is an sdhA gene;
(b) the protein involved in polyhydroxyalkanoate or PHB biosynthesis and/or granule homeostasis comprises a Granule-Associated Protein (GAP), a phasin, a PHB synthase, Gap11, Gap 20, PhaC, or PhaR;
(c) the succinyl-CoA synthetase comprises SucC and/or SucD;
(d) the protein involved in isocitrate synthesis comprises a citrate synthase, an aconitase, or both a citrate synthase and an aconitase; or
(e) the protein involved in the EMC pathway comprises: a protein that catalyzes the synthesis of acetoacetyl-CoA from acetyl-CoA, a protein that catalyzes the synthesis of hydoxybutyryl-CoA (OHB-CoA) from acetoacetyl-CoA, a beta-ketothiolase, an acetoacetyl-CoA reductase, an NADPH-linked acetoacetyl-CoA reductase, or any combination thereof.

57. The microorganism of claim 56, wherein:
(d) said citrate synthase is gltA and/or said aconitase is acnA; or
(e) said beta-ketothiolase is PhaA and/or said acetoacetyl-CoA reductase is PhaB.

58. The microorganism of claim 53, further comprising one or more of the following:
(1) overexpression of one or more serine-cycle enzymes through modifications to their corresponding genes;
(2) heterologous expression of one or more genes involved in succinic acid production;
(3) incorporation of genetic switch(es);
(4) modifications allowing accumulated PHB carbon to be made available for succinic acid production; and
(5) inhibition/inactivation of one or more gene(s) encoding succinate dehydrogenase paralogues and/or orthologues.

59. The microorganism of claim 58, wherein:
(1) the serine-cycle enzymes comprise a hydroxymethyltransferase, an enolase, and/or a malate dehydrogenase;
(2) the one or more genes involved in succinic acid production encode a pyruvate carboxylase, a phosphoenol pyruvate carboxylase, and/or an isoctrate lyase;
(3) the genetic switch(es) comprise sRNAs-, cumate-, CymR- and/or cTA-dependent genetic switch(es);
(4) the modifications allowing accumulated PHB carbon to be made available for succinic acid production comprise genes encoding PHB depolymerases and/or recycling enzymes; or
(5) the succinate dehydrogenase paralogues and/or orthologues comprise L-aspartate oxidase and/or a succinate dehydrogenase flavoprotein subunit.

60. A method for preparing succinic acid or a salt thereof, said method comprising growing the microorganism as defined claim 53 in the presence of one or more C1-compound(s) under conditions enabling the production of succinic acid or a salt thereof.

61. The method of claim 60, wherein said C1-compound comprises methane or methanol.

62. The method of claim 60, further comprising supplementation with malic acid or a salt thereof during cultivation.

63. The method of claim 60, wherein the microorganism is grown without additional supplementation with malic acid or a salt thereof during cultivation, other than malic acid added initially to the culture media.


SEQUENCE LISTING (22 sequences)

SEQ ID NO: 1 - DNA, 4367 bp, Artificial Sequence
  pCHOI2 vector
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgatccatatgtaagatttaaatgcaaccgttttttcggaaggaaatgatgacctcgtttccaccgaattagcttgcatgcctgcagatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgccaggacgaggcagcgcggctatcgtggctggccacgacgggcgttccttcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgcaggtcgactctagatcacctccttaagcttcccgcttggtcgggccgcttcgcgagggcccgttgacgacaacggtgcgatgggtcccggccccggtcaagacgatgccaatacgttgcgacactacgccttggcacttttagaattgccttatcgtcctgataagaaatgtccgaccagctaaagacatcgcgtccaatcaaagcctagaaaatataggcgaagggacgctaataagtctttcataagaccgcgcaaatctaaaaatatccttagattcacgatgcggcacttcggatgacttccgagcgagcctggaacctcagaaaaacgtctgagagataccgcggatccaattgcaatgatcatcatgacagatctgcgcgcgatcgatatcagcgctttaaatttgcgcatgctagctatagttctagaggtaccggttgttaacgttagccggctacgtatactccggaatattaataggcctaggatgcatatggcggccgcctgcagctggcgccatcgatacgcgtacgtcgcgaccgcggacatgtacagagctcggtaccatcgatagatccgttcagggcatcaaatgcagactcgtttctgacacttgcagactggatctatctcatctgcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 2 - DNA, 5560 bp, Methylobacterium extorquens
cgtgccgaacctcgtcatggctggcactgccgagacgctgttcgatatccatctcagctatttgccgtatctctttctgcacgctccggttctcggcctcgtcaagggcgcgctgctcgtcgcctgcacgatcctgatctttcccgatcgcctcgacgggaagcggcccgaactgccgccgctgccccccttatcggccgaagagcggcgcctctcggtgatcctcgcggtgatgctggcgctgtggctcagcgacggctggcatggcatcccgccggcctggatcgggctcgcggccgcggtgatctgcctgctgccgggcgtcggcgtcctgtcgtcggcgagtttcggtgagatcccgttgcggacctgcttctacgtcgccgccctgttcgggctgacggcggtggtcaacgaaaccgggctcggcgccgatctcggccacgcgctcctctcggtcgccccgctggaaccgggcgcgcccgccaagaacttcgcgaccctgaccggcctgtcgatcggcttcctgttcaccgccaccgccaacggcgcgcccgccctctacacggcgctggcagaggagttctcgcgggccagcggcttcgacctgatgagcgtactgatggtgcaggtggtcggctattcgacgctgttcctgccttatcaggcgccgcccatcatcatgacgatggatctcggccgcctgtccctggccgacgcgaccaaactcacgctcgccacgggcgtcgcgtcgctgctggtggcgaccccgcttgcctatttctggttcaggctgaccgggagactggcatgagacagatccgcgcgatcgtcagcttgaccaatgctgcaccgcagcaccggcattgccggtcggagccgagcgccctaacccttccctggaatttatccagtcttcgacgcgaggatcgtgagccgcgatgaaccctaccgtcagacctacggtcgcccagccgctctcgccccatctccagatctaccgctggacctggaccatggcgatgtcggtgttccaccgcatcaccggcacggcgctctatggcggcaccttcctcgtcgcggtgtggctggtggcgctcgcctccggtccgcgcgcctacgacacggtggcgtggttcttcggttcgtgggccgggcgcgccatcctgttcgcctatacctgggtgctgctgcaccacatgctcggcggcctgcgccacttcatctgggatgcgggctacggcttcgaccgcgaccgtcgcctggggctcgcccgcgccacgctgatcggctccgtgaccctgaccatcgtgatctgggcgatcgccctgctcgtgcgctgaggctcgccatgtcccaggtcgacaaccgccagaacaccgcgctctcgatgcgcacgccccgcgcccgggtgaagggcctcggcgcttcgggccacggcgccggccatttctggctgcagcgcctgaccggcgcctcgaacgcgctcctgatgctcgccttcgtcgtgatcatcgcgctgatggccgggcggacctacccgcaggcggtcgcgctggtgtcgcacccgctggttgcgatcatcctgatcctggccgtggtgtcggtgacgatccacatgcgtctggggatgcagacggtcatcgaggactacgtccacagccacggcctcaagttcgcggcgctcatcgccaacaccttctacgcggtcgcggtggccgccgcctgcctctacgcgatcctccgcgtgagcctgggcggcctcgcctgatcgtcgtcacggaaccaaggattcacgccatggcctccaacggaacgaacggcggcgcgcccgcctacgccatcaccgaccacaccttcgacgtggtgatcgtcggcgccggcggcgcgggtctgcgcgccacggtcggctgctcccaggccgggcttcgcaccgcctgcatcaccaaagtcttcccgacccgctcgcacaccgtggcggcgcagggcggcatctccgcctcgctcggcaatatggggccggacgattggcgctggcacatgtacgacaccgtgaagggctcggactggctcggcgaccaggacgcgatcgagtacctcgttcgcaatgcgcccgccgccgtctacgaactcgagcattggggcgtgccgttctcccgcacggacgagggcaagatctaccagcggcccttcggcggcatgaccaccgattacggcaagggcactgcccagcgcacctgcgccgcggccgaccgcaccggccatgccatgctccacacgctctacgggcaggcggtgaagaacaagacccaattcttcatcgagtacttcgctctcgacctgatcatggacgaggacggccgctgccgcggcgtgatggcgatcgatcaggccactggcgagatccaccgcttccgcgcccagcagaccatcctcgccaccggcggctacggccgcgcctacttctcggcgacctcggcccatacctgcaccggtgacggcaacgccatggtgctgcgcgccggcctgccgctgcaggacatggagttcgtgcagttccaccccaccggcatctacggcgccggctgcctcatcacggagggcgcgcgcggcgagggcggctacctcaccaactccgagggcgagcgcttcatggagcgctacgccccctcggccaaggacctcgcctcccgcgacgtggtctcacgctccatgaccatggagatccgcgacggccgcggcgtgggcaaggaaggcgaccacatcttcctgcatctcgaccatctcgacccgaagatcctgcacgagcgcctgccgggcatctcggaatcagcaaagatcttcgcgggcgtggacgtgaccaaggagccgatcccggtcctgccgacggtccactacaacatgggcggcatcccgacgaacttccacggcgaggtgctgaccctcaagaacggcaaccccgacacggtggtgccgggcctgatggcgctcggcgaggcggcctgcgtctcggtgcacggcgccaaccgcctcggctcgaactcgctgatcgacctcgtggtgttcggccgcgccgcgggcctgcgctgcgccgacatcgtcgagcccgacgcccgccagccggaactgccgaagggctcggccgacaaggcgctctcgcgcctcgaccgcttccgctacgccgacggcggcaccccgaccgcgcagctgcgcgaccggatgcagcggacgatgcagaacaactgcgccgtcttccgcaccggcgaggtgctggaggagggcaagggcctgatccacgaagtctggcgcggcgtctccgacatcgcgatcaccgaccgttcgctggtgtggaacaccgacctgctcgagaccctggaattcgacaacctgatcggccaggcggtcgtgactatggaatcggcggtcaaccgcaaggaaagccgcggcgcgcatgcccgcgaggacttccctgaccgcgacgataaggagtggatgaagcacacgctcgcgtggctcgaccaatccaagcacggcgtcgagatcgattaccgcccggtccacacctacacgatgtcgaacgacatccagtacatcgagccgaaggcgcgggtgtactgatgcagcccgcggcgcagtcggtcgaacggggaggggcggggacgatgatcccagtctcgcgtcgcctcgtgcgtgccgtcctgtccgtcgcggcggggttgctgctgatcgtgccggcagcggcgcgggcaccggatttcgacttcccggacgaagtcgcgggcttcagtcgcggcccccgcacggattacgaggcccgggcaccgggactcggctactccgtcgtctacacgaacgggcgctggaaggcggacatctacgtctacgatgccggtgtggcgaacatccccgacggagcgtcctcaccggtggtcaccggacagctcgcgcaggcggccggcgacgtcaggacggcggtggcgcagggcatctaccgcggcagcgaggatcgcggcgcggagcgcctgccgggggaggcgggcgcccggctcacctgccgcgccttttccatcgatcacccggccctcggccccaccgagagcctgctctgcctcaccggtctgcgcggccaattcgtgaagttccgcctgtccggcccggccgcgaagcagcctccgaaggcggaggccgagcggttcatgaccggctggctcgaccggcaataggatcttcgaaactcggagcgcgccgcaaggccgccgcacctcaaaaggcacccgacatggcccagttcaatcttcccaagaactcccaggtcaccgaaggccggacctggccggcgccggacggcgccaggaacctccagacgttccggatctatcgctggaacccggatgacgggaagaacccgcgcatcgacacctatagggtcgatcgcgacgattgcggcccgatggtcctcgacgccctgctgtggatcaagaacaaggtcgatccgacgctggtcttccgccgctcctgccgcgagggcatctgcggttcctgcgccatgaacatcgagggccagaacgcgctcgcctgcaccatgggcatcgacgagtgcaagacccctgacaacgccctgccgttcctgacccgcaaggacaaggacggggcggtgcggatctacccgctgccgcacatgccggtcctgaaggacctcgtgccggatctgaccaacttctacgcacagcacgcgtcgatcgagccctggctccagaccgagacgcccgcgcccgagaaggagtggaagcagacgcccgaggaccgcgcccggctcgacggcctctacgagtgcatcctgtgcgcctgctgcaccaccagctgcccgagctactggtggaacggcgacaagttcctcggcccggcagccctgcttcaggcctatcgctggctgatcgacagccgcgacgagaacaccggtgagcgcctcgacgggctgcacgatccgttccggctctaccgctgccacacgatcatgaactgcgccaacacctgcccgaagaacctcaatccggccaaggcgatcgccgagatcaagaagatgatggtcgaacgcgaagtctgatcgcttcgccgcaggaaacccttcgagacgacgttcgagagccgcgccccaccctggcgcggctctcctcgttccggcactccacaggccgtgctcatcgactgaggcaggcccgccacactgccgcgaaaacctggacgtgtacggcttttccccggcccggcgtgcagcctgccttgccgcaagcgcatttggccctgagggcaagcgcgaccgttgcgtgtttggggtgaggggtctttgatgacgcgtgggttcctgtcgaaagccgcctgtctcgcgctggccgcgagcgtcggcgcctgcgcctccaaccgcttcgaggggccccgcgagcgtccgcggccccaggcggcgctcgaacccgccacgcccgcgctgcccgccggcacggtgacgagcgagccgctcgcgcccccgccgggcgcctccgccgccccgggcgagccgccgccc

SEQ ID NO: 3 - DNA, 1441 bp, Methylobacterium extorquens
gagccgcgaggacggtgccgtccgaaccggcgacgccggcccgcagcgggagcgccgcaccggcggcgtagaggatgcgttcggctgcctcgtcgagaacgagcagagcgctgccctcgcgaacgagatgcgcgatcgatgcatcgtctctccaacgcgccaacgcgtcacgaatggccgcatcgtcgcctggataggaagcctgcactgcgcggtccgacatccgaaccctggtcatgaaccccttcatcgccctgtcgccggtcgttcggggcatgaacacctgagccctgtgtaacttaactgcggccagacgtggacactgaagcctgccgccacacgtttatcaactggtaacctcaattggtcccccggggctggaaaaaaggttacccctgcgttattgtgcagtgcacattgtgcgccgcacaacgagcgcccgatgccaacatcaggagagagacgtgacaaacactccgaattacgaagtcccgaccgagatgcgcgacttcgccgagaagagcgtcgagcaggcccgtaaggcgttcgattcgttcatcggcgcggcccgccgcaccgccgacaccgtccagggctcgaccgacctcgcccgcagcaacgccaccagcatctcctcgcgcggcttcgagtacgctgagcagaacgtcaacgccgcgttcgacctggcgcagaagctcgtgcgctcgcgcgatgtgcaggaggccatgcagcaccaggcggagttcgtgcgcgcgcagttcgcggcgattcaggcgcaggccaaggagttcggtggcctcgcacagagcgcctttcagcagagcgccgagaacgccaagagcgtcatgcagcagggtgccgccgacgctcgccaagcctacgagcagggcgtcgagacagcccgcgagaatgccaacgacgcgcagaaatcttcttcctgacgcaccctccgcactacggttcggatggcacgtaaggccgacggcccgctcccaacggagtgggccgtcctcgtttcgggcttcgtccctttggttttatccagaaaagcggtctcccgctccgcctcttttccgccgccggggcaccgcaaagctccccgcctcgccggctccgtgagaggcggcccagcggaggttttggacacgatgcgtcccgttcgtactctcggtctcgcggccctgatcgccgtcgtggccggaagcctcgcgcccgcccaggcacaatattacgacgacggctacgatgcgcctccgccccgcgcgcgcacctatcgcgatcgcgattggggtggcccgccggcgcgccgagccatgggcctcaattgcgatgcggtgcaatcgggcatcagcggcctgcagccgttctcctgccctctgcccggcccgcgcccgctgggcgcccgctgcttctgcgatatgccggtgtcgttcctgaacgg

SEQ ID NO: 4 - DNA, 2093 bp, Methylobacterium extorquens
atgaacatccacgaataccaggcaaaggctgtcctgaaggaattcggcctgccggtctcccgcggcgtgccgatcttcaagccggaggaggccgctgcggcggccgacgaactcggcggcccggtctgggtggtgaagtcgcagatccatgcgggcgggcgcggcaagggcaccttcaagggcgcgcccgagggcgccaagggcggtgtgcgcgtcaccaagtccaaggacgaggtggtgcagttcgctcgcgaaatgctcgggcagacgctcgtgaccgtgcagaccggcgaggccggcaagcaggtcaaccgcctctacatcgaggagggcgcggcgatcgccgccgagttctacctgtcgatgctggtcgatcgcgagaccggccgggtcgccttcgtcgtctcgacggagggcggcatggatatcgagcaggtcgcccacgacacccctgagaagatcgtcaccttctcggtcgatccggccaccggcatcatgccccatcacggccgggccgtcgccaaggcgctcggcctctccgggccccaggccaaggaagcgggcgcgctcaccgagaagctctacgcggccttcacggccaaggacatgagcatgctggagatcaacccgctggtgctcaccggcgacggcggtctcaagtgcctcgacgccaagatctcgttcgattcgaacgccctctaccgccaccccgagatcgtcgcgctgcgcgacgagaccgaggaggacgccaaggagatcgaggcctcgaaatacgacctcgcctacatcgccctcgacggtaccatcggctgcatggtcaatggcgccggcctcgccatggcgaccctcgacatcatcaagctctacggcgaggagccggcgaacttcctcgatgtcggcggcggcgcctccgaggagaaggtcacggcggccttcaagatcatcaccgccgatccgcaggtgaagggcatcctcgtcaacatcttcggcgggatcatgaagtgcgacgtcatcgcccgcggcgtgatcgcggcggtgaaggcggtcggcctcgaagtgccgctcgtcgtgcgtctggaaggcacaaacgtcgaggagggcaaggagatcatccgaaactccggcctcaacgtgatccccgcggacgatctcgacgacgccgcgcagaagatcgtcgccgcggtgaagaagggttgagctgacagatgtccattctcatcgaccagaacaccaaggtcatctgccagggcttcaccggcaagaacggcacgttccactccgagcaggccatcgcctacggcaccaagatggtcggcggcacgagcccgggtaagggtggctccacccatctcggactgccggtgttcgataccgtggccgaggcgcgcgaggccaccggcgccgacgcctccgtcgtctacgtgccgccgccgggcgcggccgatgcgatctgcgaggcgatccaggcggagatcccgctgatcgtctgcatcaccgagggcattccggtgctcgacatggtgcgggtcaagcgcgcgctcgaaggctcgaagtcgcgcctgatcgggccgaactgccccggcgtcgtcaccgcgggccagtcgaagatcggcatcatgccggccaacatcttcaagccgggttcggtcggcatcgtctcgcgctccggcacgctgacctacgaggcggtgttccagaccaccgtcgaggggctcggccagaccaccgccgtcggcatcggcggcgatcccgtcaagggcaccgagttcatcgccatgctggagctgttcctggccgatcccaagaccgagtcgatcgtgatgatcggcgagatcggcggctcggccgaggaagaggcggcacagttcctggccgacgaggccaagcgcggccgcaagaagccgatggtcggcttcattgccggccgcaccgcgcctcccggccgccgcatgggccatgccggcgcgatcatctccggcggcaagggcggcgccgaggataagatcgcggcgatggaggccgcgggcatccgcgtctcgccttcgccggcccgtctcggctccacgctcgtggacgtgctgaagtccaggtga

SEQ ID NO: 5 - DNA, 24 bp, Artificial Sequence
  primer
gaattcctgatgctcgccttcgtc

SEQ ID NO: 6 - DNA, 25 bp, Artificial Sequence
  Primer
gcggccgctgctcgagttcgtagac

SEQ ID NO: 7 - DNA, 23 bp, Artificial Sequence
  Primer
gggcccgtcgtgaccatggaatc

SEQ ID NO: 8 - DNA, 20 bp, Artificial Sequence
  Primer
gagctcgctgccgcggtaga

SEQ ID NO: 9 - DNA, 23 bp, Artificial Sequence
  Primer
ctgcagatgattgaacaagatgg

SEQ ID NO: 10 - DNA, 26 bp, Artificial Sequence
  Primer
ctgcagtcagaagaactcgtcaagaa

SEQ ID NO: 11 - DNA, 26 bp, Artificial Sequence
  Primer
ggatccatgaacatccacgaatacca

SEQ ID NO: 12 - DNA, 24 bp, Artificial Sequence
  Primer
ggtacctcacctggacttcagcac

SEQ ID NO: 13 - DNA, 19 bp, Artificial Sequence
  Primer
cgagaagactgtctcgaac

SEQ ID NO: 14 - DNA, 19 bp, Artificial Sequence
  Primer
catcgcgattgtcgattcg

SEQ ID NO: 15 - DNA, 33 bp, Artificial Sequence
  Primer
aaaaaaggatccatgaccgcgatcaccaatatc

SEQ ID NO: 16 - DNA, 32 bp, Artificial Sequence
  Primer
aaaaaagctagcatgcttcaggtgcgatcagc

SEQ ID NO: 17 - DNA, 21 bp, Artificial Sequence
  Primer
atgttggcgaagccctccttc

SEQ ID NO: 18 - DNA, 20 bp, Artificial Sequence
  Primer
gattcggcgagcaccattcc

SEQ ID NO: 19 - DNA, 24 bp, Artificial Sequence
  Primer
ggatccacacgtcctcccaaaggt

SEQ ID NO: 20 - DNA, 24 bp, Artificial Sequence
  Primer
ggatcctgaaggtgtgagggatcg

SEQ ID NO: 21 - DNA, 27 bp, Artificial Sequence
  Primer
ggatccgcataacttcgtatagcatac

SEQ ID NO: 22 - DNA, 23 bp, Artificial Sequence
  Primer
gataagctggatccataacttcg